(19) United States
(12) Patent Application Publication
(10) Pub. No.: US 2021/0077413 A1
(43) Pub. Date: Mar. 18, 2021

(54) CHOLESTYRAMINE FORMULATIONS AND METHODS OF USE

(71) Applicant: PHARMASCIENCE INC., Montreal (CA)

(72) Inventors: Sophie Tanguay, Montreal (CA); Smita Debnath, Montreal (CA); Mustapha Kandil, Montreal (CA)

(21) Appl. No.: 16/965,297

(22) PCT Filed: Jan. 30, 2019
(86) PCT No.: PCT/CA2019/050116

§ 371 Date: Jul. 27, 2020

Related U.S. Application Data
(60) Provisional application No. 62/623,895, filed on Jan. 30, 2018.

(51) Int. Cl. A61K 9/48; A61K 31/785

(52) U.S. Cl. CPC A61K 9/4891; A61K 31/785; A61K 9/4866; A61K 9/4858; A61K 9/485

(57) ABSTRACT

The present disclosure relates to cholestyramine formulations for targeted delivery to the ileum, its use and its process of manufacture, more particularly scale-up manufacture.

### BACKGROUND

Bile acid malabsorption (BAM) is a condition characterized by an excess of bile acids in the colon, often leading to chronic diarrhea. Primary bile acids are steroid acids that are synthesized from endogenous cholesterol and conjugated in the liver. From the liver, they are excreted through the biliary tree into the small intestine where they participate in the solubilization and absorption of dietary lipids and fat-soluble vitamins. When they reach the ileum, most bile acids are reabsorbed by active transport in the ileum into the portal circulation and returned to the liver for further secretion into the biliary system.

A small proportion of the secreted bile acids is not reabsorbed in the ileum and reaches the colon. Here, bacterial action results in deconjugation and dehydroxylation of the bile acids, producing the secondary bile acids such as deoxycholate and lithocholate. In the colon, bile acids (in particular the dehydroxylated bile acids chenodeoxycholate and deoxycholate) stimulate the secretion of electrolytes and water. This increases the colonic motility and shortens the colonic transit time.

If present in excess, bile acids produce diarrhea or bile acid diarrhea (BAD) with other gastrointestinal symptoms such as bloating, urgency and fecal incontinence. There have been several recent advances in the understanding of this condition of bile salt or bile acid malabsorption, or BAM (Pattni and Walters, Br. Med. Boll. 2009, vol 92, p. 79-93; Islam and Di Baise, Pract. Gastroenterot. 2012, vol, 36(10), p. 32-44). Dependent on the cause of the failure of the distal ileum to absorb bile acids, bile acid malabsorption may be divided into Type 1 (Ileal dysfunction and impaired reabsorption, e.g., Crohn's disease), Type 2 (primary, or idiopathic, BAD produces a similar picture of increased fecal BAs, watery diarrhea, and response to BA sequestrants in the absence of ileal or other obvious gastrointestinal disease) and Type 3 BAM (other gastrointestinal disorders which affect absorption, such as small intestinal bacterial overgrowth, celiac disease, or chronic pancreatitis).

Diarrhea may also be the result of high concentrations of bile acid in the large intestine following treatment with drugs that increase the production of bile acids and/or influence the reabsorption of bile acids by the small intestine, such as treatment with ileal bile acid absorption (IBAT) inhibitors.

Diarrhea may also be the result of Short bowel syndrome (SBS, or simply short gut) which is a malabsorption disorder caused by a lack of functional small intestine. The primary symptom is diarrhea, which can result in dehydration, malnutrition, and weight loss. Other symptoms may include abdominal pain, bloating, heartburn, steatorrhea, fatigue, lethargy, lactose intolerance, and foul smelling stool. Complications can include anemia and kidney stones.

Most cases are due to the surgical removal of a large portion of the small intestine. This is most often required due to Crohn's disease in adults and necrotizing enterocolitis in young children. Other common reasons for extensive resection are mesenteric infarction, radiation enteritis, cancer, volvulus, trauma and congenital anomalies.

Treatment may include a specific diet, medications, and/or surgery. The diet may include slightly salty and slightly sweet liquids, vitamin and mineral supplements, small frequent meals, and the avoidance of high fat food. Occasionally nutrients need to be given through an intravenous line, known as parenteral nutrition. There are estimated to be about 15,000 people with the condition in the United States.

The current medicinal treatment of bile acid malabsorption aims at excreting bile acid in the feces by first binding excess bile acids in the gastrointestinal tract, beginning in the proximal part of the small bowel, thereby reducing the secretory actions, of the bile acids. For this purpose, cholestyramine is commonly used as the bile acid sequestrant. Cholestyramine (or colestyramine, CAS Number 11041-12-6) is a strongly basic anion-exchange resin that is practically insoluble in water and is not absorbed from the gastrointestinal tract. Instead, it absorbs and combines with the bile acids in the intestine to form an insoluble complex. The complex that is formed upon binding of the bile acids to the resin is excreted in the feces. The resin thereby prevents the normal reabsorption of bile acids through the enterohepatic circulation, leading to an increased conversion of cholesterol to bile acids to replace those removed from reabsorption. This conversion lowers plasma cholesterol concentrations, mainly by lowering of the low-density lipoprotein (101)-cholesterol.

Cholestyramine is also used as a hypolipidemic agent in the treatment of hypercholesterolemia, type II hyperlipoproteinemia and in type 2 diabetes mellitus. It is furthermore used for the relief of diarrhea associated with SBS, ileal resection, steatorrhea (fat stool), Crohn's disease, vagotomy, diabetic vagal neuropathy and radiation, as well as for the treatment of pruritus in patients with cholestasis, who suffer from bile acid deposited in the dermal tissue.

Cholestyramine 2 to 4 g taken daily with meals reduces diarrhea associated with bile acid malabsorption due to ileal resection. According to U.S. labeling, the recommended starting adult dose for the reduction of elevated serum cholesterol, is 4 g of cholestyramine once or twice a day. The recommended maintenance dose is 8 to 16 g divided into two doses. It is recommended that increases in dose be gradual with periodic assessment of lipid/lipoprotein levels at intervals of not less than 4 weeks. The maximum recommended daily dose is 24 g. Although the recommended dosing schedule is twice daily, cholestyramine may be administered in 1 to 6 doses per day. In the treatment of pruritus, doses of 4 to 8 g are usually sufficient. The most common side-effect is constipation, while other gastrointestinal side-effects are bloating, abdominal discomfort and pain, heartburn, flatulence and nausea/vomiting. There is an increased risk for gallstones due to increased cholesterol concentration in bile. High doses may cause steatorrhea by interference with the gastrointestinal absorption of fats and concomitant decreased absorption of fat-soluble vitamins (A, D, E, K). Chronic administration may result in an increased bleeding tendency due to hypoprothrombinemia associated with vitamin K deficiency or may lead to osteoporosis due to impaired calcium and vitamin D absorption. There are also occasional reports of skin rashes and pruritus of the tongue, skin and perianal region. Due to poor taste and texture and the various side effects, >50% of patients discontinue therapy within 12 months.

Another drawback with the current treatment using cholestyramine is that this agent reduces the absorption of other drugs administered concomitantly, such as estrogens, thiazide diuretics, digoxin and related alkaloids, loperamide, phenylbutazone, barbiturates, thyroid hormones, warfarin and/or some antibiotics. It is therefore recommended that other drugs should be taken at least 1 hour before or 4 to 6 hours after the administration of cholestyramine. Dose adjustments of concomitantly taken drugs may still be necessary to perform.

In view of these side effects, it would be desirable if cholestyramine could be formulated for later release in the gastrointestinal system, i.e. the ileum. Such a formulation may require a lower dose of cholestyramine and should have better properties regarding texture and taste, and may therefore be better tolerated by the patients. More importantly, ileal release of cholestyramine should reduce or eliminate interactions with other drugs and should lower risks for malabsorption of fat and fat-soluble vitamins, while still binding bile acids in order to reduce the increased colonic secretion and motility. For reasons of patient compliance, it would furthermore be desirable if the number of unit dosage forms (UDF) to be taken could be kept as low as possible. Each UDF should therefore contain as much cholestyramine as possible, taking into account that the dosage form should not be too large for comfortable administration.

Jacobsen et al. (Br. Med. J. 1985, vol. 290, p. 1315-1318) describe a study wherein patients who had undergone ileal resection were administered 500 mg cholestyramine tablets coated with cellulose acetate phthalate (12 tablets daily). In five of the 14 patients in this study, the tablets did not disintegrate in the desired place.

US2003/0124088 discloses preparations for preventing bile acid diarrhea which comprise containing a bile acid adsorbent such as cholestyramine coated with a polymer so as to allow the release thereof around an area from the lower part of the small intestine to the cecum.

US2017/0224721 discloses an oral formulation for targeted delivery of cholestyramine to the colon, comprising a plurality of cholestyramine pellets that are coated with a colon release coating, and the use of this formulation in the treatment of bile acid malabsorption.

European patent application EP 0040590 discloses an oral pharmaceutical preparation comprising a core containing a therapeutically active substance and a coating, characterized in that the coating comprises an anionic carboxylic acrylic polymer soluble only above pH 5.5 in an amount of 10 to 85% by weight of the coating and a water-insoluble polymer selected from a quaternary ammonium substituted acrylic polymers in an amount of 15 to 90% by weight of the coating. The preparation is said to release a major part of the drug contents thereof in the lower part of the intestinal system.

US 2011/0294767 (Gedulin et al.) discloses compositions comprising bile acid recycling inhibitors and/or enteroendocrine peptide enhancing agents in association with a matrix that allows for controlled release in the distal part of the ileum and/or the colon for the treatment of obesity, diabetes and inflammatory gastrointestinal conditions. The time released formulation may comprise a capsule with hydrogel plug.

US 2013/0034536 (Gedulin et al.) discloses compositions comprising bile acid recycling inhibitors and/or enteroendocrine peptide enhancing agents in association with a matrix that allows for controlled release in the distal part of the ileum and/or the colon for the treatment of pancreatitis.

US 2013/0236541 (Gillberg et al.) and US 2017/0182115 (Gillberg et al.) disclose pharmaceutical combinations comprising an ileal bile acid transport system (IBAT) inhibitor and a bile acid binder for the treatment of a cholestatic liver disease such as ALG, PFIC, PBC or PSC.

Despite progress made in this area, there still is a need for further improved cholestyramine formulations. In particular, there is a need for oral formulations for targeted delivery of cholestyramine to the ileum.

### SUMMARY

Because of its physico-chemical properties (presence of quaternary ammonium functional groups, hygroscopic properties, etc.) cholestyramine interacts with other polar/charged molecules (including some enteric coatings). Under certain conditions, it tends to aggregate and/or create static on manufacturing surfaces, thereby complexifying the manufacturing process and reducing manufacturing yield.

Stable, high quality, enteric coated capsules are difficult to make especially at large scale due to the difficulty in obtaining even (uniform) coatings on all capsules, particularly near the seam where the two parts of the shell are joined. Furthermore, large scale manufacturing processes tend to damage the fragile enteric coating on the capsule and affect the quality of the final product and release of the active ingredient(s). Accordingly, there are very few drugs on the market which are enteric coated capsules. Generally, the content of the capsule (minitabs or pellets) are coated, not the capsule itself. In such case the capsule shell helps to protect the enteric coating.

By selecting the right combination of reagents, it was possible to develop a stable enteric coated capsule formulation, suitable for large scale preparation, having the desired release profile and which was shown to significantly reduce interactions between cholestyramine and other drugs, particularly those absorbed in the proximal part of the small intestine.

The present disclosure thus relates generally to cholestyramine formulations for targeted delivery to the ileum.

In a preferred embodiment, a UDF is in the form of a capsule which comprises a coating comprising one or more enteric polymers. The formulations may be used for any condition amenable to cholestyramine treatment. The formulations are preferably used for treatment of bile acid diarrhea, preferably in patients with SBS (e.g., type I, type II and/or type III BAM).

Accordingly, the present description relates to the following implementations:
1. A unit dosage form comprising a capsule having a coating comprising one or more enteric polymers and a capsule fill comprising cholestyramine.
2. The unit dosage form of implementation 1, wherein the capsule has the following in vitro dissolution profile using a USP Type 3 apparatus: (a) pH 1.2, no disintegration of capsule for 1 hour; and (b) pH 6 to 7, preferably 6.2 to 7, more preferably 6.5 to 6.8, or 6.5, 6.6, 6.7 or 6.8 bursting of capsule and dispersion of the capsule contents within 30 minutes, wherein (a) is performed prior to (b).
3. The unit dosage form of implementation 1 or 2, which also has the following in vitro dissolution profile using a USP Type 3 apparatus: (a) pH 1.2: no disintegration of capsule for 1 hour; (b) pH 6.2-7, preferably 6.5-6.8, preferably 6.5 or 6.8: bursting of capsule and dispersion of the capsule content within 30 mins, and (c) pH 6.0, bursting of capsule and dispersion of capsule content after at least 45 mins, wherein (a) is performed prior to (b) or (c).
4. The unit dosage form of implementation 1, having the following in vitro dissolution profile using a USP Type 3 apparatus: (a) pH 1.2: no disintegration of capsule for 1 hour; (b) pH 6.8: bursting of capsule and dispersion of the capsule content within 20 mins, preferably within 15 mins; and (c) pH 6.0, nor bursting or bursting of capsule and dispersion of capsule content after at least 45 mins, wherein (a) is performed prior to (b) or (c).
5. The unit dosage form of any one of implementations 1 to 4, wherein the unit dosage form starts releasing the capsule fill at pH>6, preferably at pH>6.2.
6. The unit dosage form of any one of implementations 1 to 5, wherein in vivo the unit dosage form starts releasing the capsule fill after the proximal part of the small intestine.
7. The unit dosage form of any one of implementations 1 to 6, wherein in vivo the unit dosage form releases the capsule fill in the distal part of the small intestine, preferably in the jejunum, more preferably in the ileum.
8. The unit dosage form of any one of implementations 1 to 7, wherein the capsule fill comprises at least 70% w/w cholestyramine, more preferably at least 75% w/w cholestyramine, more preferably at least 80% w/w cholestyramine, even more preferably at least 83% w/w cholestyramine, and most preferably at least 85% w/w cholestyramine.
9. The unit dosage form of any one of implementations 1 to 8, wherein the amount of cholestyramine in the unit dosage form ranges from 350 to 500 mg, more preferably 400 to 450 mg, most preferably 425 mg.
10. The unit dosage form of any one of implementations 1 to 9, wherein the capsule comprises gelatin, agar, xanthan gum, karaya gum, locust bean gum, gum arabic, pullulan, methyl cellulose, hydroxyethyl cellulose, hydroxypropyl methylcellulose, sodium alginate, or combinations thereof, preferably HPMC.
11. The unit dosage form of any one of implementations 1 to 10, wherein the capsule is a size 0, 00 or 000 capsule, more preferably size 00.
12. The unit dosage form of any one of implementations 1 to 11, wherein the capsule fill comprises, consists essentially of, or consists of from 80 to 90% w/w, preferably 83 to 85% w/w cholestyramine, from 10 to 20% w/w of a bulking agent, from 0.25 to 2% w/w of a glidant and from 0.25 to 2% w/w of a lubricant, the total of the fill being 100% w/w.
13. The unit dosage form of any one of implementations 1 to 12, wherein the enteric polymer coating level comprises between 5% and 15%, between 6% and 15% w/w of the UDF, preferably between 5.5% and 10%, most preferably between 5.8% and 6.8%, and even more preferably between 6.0 and 6.5%.
14. The unit dosage form of any one of implementations 1 to 13, wherein the enteric polymer coating is an acrylate or acrylic acid polymer or co-polymer, optionally comprising one or more ammonio methacrylate copolymers.
15. The unit dosage form of any one of implementations 1 to 14, wherein the enteric polymer coating is in the form of an acrylic resin lacquer in the form of an aqueous dispersion, preferably copolymers of acrylic and methacrylic esters with a low content of quaternary ammonium groups.
16. The unit dosage form of any one of implementations 1 to 15, wherein the enteric polymer coating is selected from the group consisting of co-polymers based on polymethacrylic acid and methacrylates, ethyl acrylate and methyl acrylate, co-polymers of acrylic and methacrylic acid esters, hydroxypropyl methylcellulose phthlate, hydroxypropyl methylcellulose acetate succinate, cellulose acetate phthalate, polyvinyl acetate phthalate or mixtures thereof.
17. The unit dosage form of any one of implementations 1 to 16, wherein the enteric polymer coating comprises or consists of a methacrylic acid copolymer, preferably Poly(methacrylic acid-co-ethyl acrylate) 1:1.
18. The unit dosage form of any one of implementations 1 to 17, wherein the enteric polymer coating comprises a film former, a plasticizer and/or an anti-adherent, preferably has the following formulation:

| Components | Function |
|---|---|
| Blend: | |
| Cholestyramine Resin, | Active ingredient |
| Lactose Monohydrate | Bulking agent/filler |
| Colloidal Silicon Dioxide | Glidant |
| Magnesium Stearate | Lubricant |
| Capsule Shell (Cap and Body) Size 00: | |
| Hypromellose | Capsule structure |
| Titanium dioxide | Opacifier |
| Enteric Coat: | |
| Methacrylic Acid Copolymer Dispersion | Control release polymer |
| Plasacryl ™ T20, | Plasticizer |
| Talc | Anti-adherent |

19. The unit dosage form of any one of implementations 1 to 18, further comprising a barrier coating between the capsule shell and the enteric polymer coating, preferably a seal coating.
20. The unit dosage form of implementation 19, wherein the barrier coating level comprises 6% to 15% w/w of the unit dosage form, preferably 7% to 12%, and most preferably 9% to 11%.
21. The unit dosage form of implementation 19 or 20, wherein the barrier coating level comprises a control release polymer, a plasticizer and/or an anti-adherent.
22. The unit dosage form of any one of implementations 19 to 21, wherein the capsule fill, the capsule shell, the enteric coating and/or the barrier coating further comprise excipients such as bulking agents or diluents, glidants, lubricants, and/or other common excipients.
23. The unit dosage form of implementation 21, wherein the bulking agents or diluents include, for example, dextrose, lactose, glucose, glycine, inositol, mannitol, sorbitol, sucrose, a polyethyleneglycol (PEG), or a polyvinylpyrrolidine (PVP), or a combination thereof, preferably lactose monohydrate.
24. The unit dosage form of implementation 21, wherein the glidants include, for example, calcium phosphate, calflo E, cellulose (powder), colloidal silicon dioxide, magnesium silicate, magnesium trisilicate, silicon dioxide, starch, talcum powder, or a combination thereof, preferably colloidal silicon dioxide.
25. The unit dosage form of implementation 21, wherein the lubricants include, for example, magnesium stearate, sodium stearyl fumarate, hydrogenated castor oil, hydrogenated soybean oil, polyethylene glycol, or a combination thereof, preferably magnesium stearate.
26. The unit dosage form of any one of implementations 22-25, wherein the plasticizer is Triethyl citrate.
27. The unit dosage form of any one of implementations 22-26, wherein the antiadherent is talc.
28. The unit dosage form of any one of implementations 22-27, wherein the other common excipients comprises glyceryl monostearate, and/or polysorbate 80.
29. The unit dosage form of any one of implementations 22-28, wherein (i) the capsule fill comprises in addition to cholestyramine or consists essentially of a filler, one or more glidant and one or more lubricant; (ii) the barrier coating or seal coat comprises or consists essentially of HPMC, one or more plasticizer and one or more anti-adherent; and/or (iii) the enteric coating comprises or consists essentially of a Methacrylic acid copolymer, one or more plasticizer, and one or more an-anti-adherent.
30. The unit dosage form of any one of implementations 19 to 26, wherein the enteric polymer coating and/or the sealing coating are applied onto the capsule by a fluidized bed method, a rotating fluidized bed method, a side-vented pan or coating pan method, preferably a coating pan.
31. The unit dosage form of any one of implementations 1 to 18, comprising the following components:

| Components | Function |
|---|---|
| Blend: | |
| Cholestyramine Resin, | Active ingredient |
| Lactose Monohydrate | Bulking agent |
| Colloidal Silicon Dioxide | Glidant |
| Magnesium Stearate | Lubricant |
| Capsule Shell (Cap and Body) Size 00: | |
| Hypromellose | Capsule structure |
| Titanium dioxide | Opacifier |
| Seal Coat: | |
| Hypromellose | Film former |
| Triethyl Citrate | Plasticizer |
| Talc, USP | Anti-adherent |
| Enteric Coat: | |
| Methacrylic Acid Copolymer Dispersion | Control release polymer |
| Plasacryl ™ T20, | Plasticizer |
| Talc | Anti-adherent |

32. The unit dosage form of any one of implementations 1 to 31, wherein the blend has about the following formulation:

a)

| Ingredient | % w/w |
|---|---|
| Cholestyramine | 85.02 |
| Lactose Flowlac 100 | 14.00 |
| Colloidal silicon dioxide | 0.48 |
| Magnesium stearate | 0.50 |

b)

| Ingredient | % w/w |
|---|---|
| Cholestyramine | 88.24 |
| Lactose monohydrate supertablet 11SD EP | 10.78 |
| Colloidal silicon dioxide | 0.48 |
| Magnesium stearate | 0.50 |
| Total | |

or

c)

| Ingredient | % w/w |
|---|---|
| Cholestyramine | 83.34 |
| Lactose monohydrate supertablet 11SD EP | 15.69 |
| Colloidal silicon dioxide | 0.48 |
| Magnesium stearate | 0.50 |
| Total | 100 |

33. The unit dosage form of any one of implementation 19 to 32, wherein the seal coating has about the following formulation:

a)

| Ingredients for seal coat | % w/w |
|---|---|
| HPMC E5 LV | 83.33 |
| TEC | 8.33 |
| Talc | 8.33 |
| Total | 100 |

b)

| Ingredients for seal coat (COEP-049) | % w/w |
|---|---|
| Opadry 03K19229 | 80.00 |
| Talc | 20.00 |
| Total | 100 |

c)

| Ingredients for seal coat (COEP-053) | % w/w |
|---|---|
| HPMC E5 LV | 71.43 |
| Triethyl citrate | 7.14 |
| Talc | 21.43 |
| Total | 100 |

or

d)

| Ingredients for seal coat | % w/w |
|---|---|
| HPMC E5 | 90.91% |
| Triethyl citrate | 9.09% |
| Total | 100 |

34. The unit dosage form of any one of implementations 19 to 33, wherein the enteric coating has about the following formulation:

A)

| Ingredients for enteric coat | % w/w |
|---|---|
| HPMCAS JZ160-122 | 95.0 |
| Triethyl citrate | 5.0 |
| Total | 100 |

B)

| Ingredients for enteric coat | % w/w |
|---|---|
| HPMCAS XCS47163 | 62.11 |
| Triethyl citrate (TEC) | 17.39 |
| Talc | 18.63 |
| Sodium lauryl sulfate (SLS) | 1.86 |
| Total | 100 |

C)

| Ingredients for enteric coat (COEP-051) | % w/w |
|---|---|
| Poly(methyl acrylate-co-methyl | 51.20 |
| methacrylate-co-methacrylic acid) 7:3:1 | |
| Poly(methacrylic acid-co-ethyl acrylate, | 12.80 |
| 1:1 (e.g. Eudragit L30D55) | |
| Triethyl citrate | 4.00 |
| Talc | 32.00 |
| Total | 100 |

D)

| Ingredients for enteric coat (COEP-053) | % w/w |
|---|---|
| Poly(methyl acrylate-co-methyl | 70.16 |
| methacrylate-co-methacrylic acid) 7:3:1 | |
| Poly(methacrylic acid-co-ethyl acrylate, | 28.06 |
| 1:1 (e.g. Eudragit L30D55) | |
| Plasacryl T20 | 1.77 |
| Total | 100 |

E)

| Ingredients for enteric coat (COEP-054) | % w/w |
|---|---|
| Poly(methyl acrylate-co-methyl | 57.9 |
| methacrylate-co-methacrylic acid) 7:3:1 | |
| Poly(methacrylic acid-co-ethyl acrylate, 1:1 | 23.1 |
| (e.g. Eudragit L30D55) | |
| Plasacryl T20 | 6.7 |
| Talc | 12.2 |
| Total | 100 |

F)

| Ingredients for enteric coat (COEP-055) | % w/w |
|---|---|
| Poly(methyl acrylate-co-methyl | 64.91 |
| methacrylate-co-methacrylic acid) 7:3:1 | |
| Poly(methacrylic acid-co-ethyl acrylate, | 16.23 |
| 1:1 (e.g. Eudragit L30D55) | |
| Plasacryl T20 | 6.69 |
| Talc | 12.17 |
| Total | 100 |

G)

| Ingredients for enteric coat (COEP-056) | % w/w |
|---|---|
| Poly(methyl acrylate-co-methyl | 55.96 |
| methacrylate-co-methacrylic acid) 7:3:1 | |
| Poly(methacrylic acid-co-ethyl acrylate, 1:1 | 25.18 |
| (e.g. Eudragit L30D55) | |
| Plasacryl T20 | 6.69 |
| Talc | 12.17 |

H)

| Ingredients for enteric coat (COEP-058) | % w/w |
|---|---|
| Poly(methacrylic acid-co-ethyl acrylate, 1:1 | 80.00 |
| (e.g. Eudragit L30D55) | |
| Plasacryl T20 | 12.00 |
| Talc | 8.00 |
| Total | 100 |

I)

| | COEP- | COEP- | COEP- |
|---|---|---|---|
| | 059 | 060 | 061 |
| Ingredients for enteric coat | % w/w | % w/w | % w/w |
| Poly(methacrylic acid-co-ethyl | 74.08 | 74.08 | 74.08 |
| acrylate, 1:1 (e.g. Eudragit L30D55) | | | |
| Plasacryl T20 | 18.52 | 18.52 | 18.52 |
| Talc | 7.41 | 7.41 | 7.41 |
| Total | 100 | 100 | 100 |

Or

K)

| Ingredients for enteric coat (COEP | |
|---|---|
| 071 (F1 and F2), COEP-072) | % w/w |
| Poly(methacrylic acid-co-ethyl | 74.2 |
| acrylate, 1:1 (e.g., Eudragit L30D55) | |
| Plasacryl T20 | 19.7 |
| Talc | 7.39 |
| Total | 100 |

35. The unit dosage form of any one of implementations 19 to 34, wherein the blend is a defined in implementation 32, the seal coating is as defined in implementation 33 and the enteric coating is as defined in implementation 34, wherein the combination of the blend, the enteric coating and the seal coating is any combination of any one of a) to c) for the blend, any one of a) to d) for the seal coating and any one of A) to K) for the enteric coating, respectively.
36. The unit dosage form of implementation 35, wherein the combination of the blend is combination of any one of a) to c) for the blend, with a combination of aA, aB, bC, cD, cF, cG, cH, cl, or cJ for the enteric coating and the sealing coating respectively.
37. The unit dosage form of any one of implementation 1 to 31, wherein the formulation is defined as below:

| | | |
|---|---|---|
| | | Phase II trial |
| Scintigraphy | DDI | (Scale up) |

Capsule Core

| | | | |
|---|---|---|---|
| Cholestyramine Resin | 85.00% | 85.00% | 85.00% |
| (active ingredient) | (425 mg) | (425 mg) | (425 mg) |
| Lactose monohydrate | 13.42% | 14.02% | 14.02% |
| (diluent) | (67.1 mg) | (70.1 mg) | (70.1 mg) |
| Colloidal silicon | 0.48% | 0.48% | 0.48% |
| dioxide (glidant) | (2.4 mg) | (2.4 mg) | (2.4 mg) |
| Magnesium stearate | 0.5% | 0.5% | 0.5% |
| (lubricant) | (2.5 mg) | (2.5 mg) | (2.5 mg) |
| Samarium oxide | 0.6% | — | — |

Sealing coat

| | | | |
|---|---|---|---|
| HPMC (Hypromellose) | 71.43% | 71.43% | 71.43% |
| (film former) | (44.5 mg) | (44.5 mg) | (44.5 mg) |
| Triethyl citrate | 7.14% | 7.14% | 7.14% |
| (TEC) (plasticizer) | (4.4 mg) | (4.4 mg) | (4.4 mg) |
| Talc (Lo-micron) (anti- | 21.43% | 21.43% | 21.43% |
| adherent, filler of gaps) | (13.3 mg) | (13.3 mg) | (13.3 mg) |
| Seal coating weight gain | 10% | 10% | 10% |

Enteric coating

| | | | |
|---|---|---|---|
| Methacrylic acid copolymer | 74.08 | 74.08 | 74.16% |
| (Eudragit ™ L30D-55, | (30.5 mg) | (30.5 mg) | |
| 30% solid dispersion) | | | |
| Plasacryl ™ T20 | 18.52% | 18.52% | 18.42% |
| (20% solid dispersion) | (7.6 mg) | (7.6 mg) | |
| Talc (Lo-micron) | 7.41% | 7.41% | 7.42% |
| (anti-adherent) | (3.0 mg) | (3.0 mg) | |
| Enteric coating | 6.5% | 6.0% | |
| weight gain (%) | | | |
| Enteric coating | 6.5% | 6.0% | 6.5% |
| weight gain | | | |

38. A method of treating diarrhea, comprising administering to a patient the unit dosage form of any one of implementations 1 to 37.
39. The method of implementation 38, wherein the diarrhea is bile acid diarrhea (BAD).
40. The method of implementation 37 or 38, wherein the diarrhea is chronic diarrhea.
41. The method of implementation 37 or 38, wherein the diarrhea is associated with Bile acid malabsorption (BAM), Short bowel syndrome (SBS), ileal resection, steatorrhea (fat stool), Crohn's disease, vagotomy, diabetic vagal neuropathy, radiation and/or pruritus in patients with cholestasis.
42. The method of implementation 41, wherein the diarrhea is associated with Short bowel syndrome (SBS),
43. The method of implementation 38, wherein the diarrhea is associated with Type I Secondary BAM (such as ileal resection, ileal dysfunction, impaired reabsorption, e.g. Crohn's disease or necrotizing enterocolitis), Type II Primary BAM (such as IBS-D), or Type III miscellaneous associated BAM disorders (such as post-cholecystectomy, gastric surgery, chronic pancreatitis, celiac disease, SIBO, radiation, microscopic colitis).
44. A method of treating hypercholesterolemia, type II hyperlipoproteinemia and/or type 2 diabetes mellitus, comprising administering to a patient the unit dosage form of any one of implementations 1 to 37.
45. A method of treating pruritus in a patient with cholestasis, comprising administering to said patient the unit dosage form of any one of implementations 1 to 37.
46. A method for reducing or eliminating drug interactions with cholestyramine, such as a blood thinner like warfarin (Coumadin, Jantoven); digoxin (digitalis, Lanoxin); propranolol (Inderal); a diuretic such as hydrochlorothiazide (HCTZ); thyroid hormones such as levothyroxine (Synthroid, Levoxyl, Levothroid); birth control pills or hormone replacement; seizure medicines such as phenytoin (Dilantin) and phenobarbital (Luminal, Solfoton); an antibiotic such as amoxicillin (Amoxil, Trimox, others), doxycycline (Adoxa, Doryx, Oracea, Vibramycin), minocycline (Dynacin, Minocin, Solodyn, Vectrin), penicillin (BeePen-VK, Pen-Vee K, Veetids, others), tetracycline (Brodspec, Panmycin, Sumycin, Tetracap); aspirin; duloxetine (Cymbalta); fish oil (omega-3 PUFAs); pregabalin (Lyrica); alprazolam (Xanax); acetaminophen; and/or vitamins such as Vitamin B12 (cyanocobalamin); Vitamin C; Vitamin D3; Vitamin A, Vitamin E; Vitamin K, comprising administering to a patient the unit dosage form of any one of implementations 1 to 37.
47. A method for reducing or eliminating drug interactions with cholestyramine, such as estrogens, thiazide diuretics, digoxin and related alkaloids, loperamide, phenylbutazone, barbiturates, thyroid hormones, warfarin and/or some antibiotics comprising administering to a patient who is receiving concomitant administration of a medication which is known to interact with conventional orally-administered cholestyramine, the unit dosage form of any one of implementations 1 to 37.
48. A method for preventing macronutrients and/or micronutrients absorption deficiency in the gastrointestinal system of a patient, comprising administering to a patient the unit dosage form of any one of implementations 1-37.
49. The method of implementation 48 wherein the macronutrients and the micronutrients include fat and fat-soluble vitamins.
50. The method of any one of implementations 38 to 49, wherein the daily dose of cholestyramine is 425 mg to 30 g, preferably 850 mg to 12 g, most preferably 1700 mg to 8.5 g.
51. The method of any one of implementations 38 to 49, wherein the daily dose of cholestyramine is 425 mg to 4250 mg/day, preferably 425 mg to 3400 mg/day, more preferably 425 mg/day, 850 mg/day, 1275 mg/day, 1700 mg/day, 2125 mg/day, 2550 mg/day, 2975 mg/day, 3400 mg/day, 3825 mg/day or 4250 mg/day, or ranges between any one of these amounts.
52. Use of the unit dosage form as defined in any one of implementations 1 to 37 for treating diarrhea in a patient in need thereof.
53. Use of the unit dosage form as defined in any one of implementations 1 to 37 for the preparation of a medicament for treating diarrhea in a patient in need thereof.
54. The use of implementation 52 or 53, wherein the diarrhea is bile acid diarrhea (BAD).
55. The use of implementation 52 or 53, wherein the diarrhea is chronic diarrhea.
56. The use of implementation 52 or 53, wherein the diarrhea is associated with Bile acid malabsorption (BAM), Short bowel syndrome (SBS), ileal resection, steatorrhea (fat stool), Crohn's disease, vagotomy, diabetic vagal neuropathy, radiation and/or pruritus in patients with cholestasis.
57. The use of implementation 56, wherein the diarrhea is associated with Short bowel syndrome (SBS),
58. The use of implementation 52 or 53, wherein the diarrhea is associated with Type I Secondary BAM (such as ileal resection, ileal dysfunction, impaired reabsorption, e.g. Crohn's disease or necrotizing enterocolitis), Type II Primary BAM (such as IBS-D), or Type III miscellaneous associated BAM disorders (such as post-cholecystectomy, gastric surgery, chronic pancreatitis, celiac disease, SIBO, radiation, microscopic colitis).
59. Use of the unit dosage form as defined in any one of implementations 1 to 37 for treating hypercholesterolemia, type II hyperlipoproteinemia and/or type 2 diabetes mellitus, in a patient in need thereof.
60. Use of the unit dosage form as defined in any one of implementations 1 to 37 for the preparation of a medicament for treating hypercholesterolemia, type II hyperlipoproteinemia and/or type 2 diabetes mellitus, in a patient in need thereof.
61. Use of the unit dosage form as defined in any one of implementations 1 to 37 for treating pruritus in a patient with cholestasis in need thereof.
62. Use of the unit dosage form as defined in any one of implementations 1 to 37 for the preparation of a medicament for treating pruritus in patients with cholestasis, in a patient in need thereof.
63. Use of the unit dosage form as defined in any one of implementations 1 to 37 for reducing or eliminating drug interactions with cholestyramine, such as a blood thinner like warfarin (Coumadin, Jantoven); digoxin (digitalis, Lanoxin); propranolol (Inderal); a diuretic such as hydrochlorothiazide (HCTZ); thyroid hormones such as levothyroxine (Synthroid, Levoxyl, Levothroid); birth control pills or hormone replacement; seizure medicines such as phenytoin (Dilantin) and phenobarbital (Luminal, Solfoton); an antibiotic such as amoxicillin (Amoxil, Trimox, others), doxycycline (Adoxa, Doryx, Oracea, Vibramycin), minocycline (Dynacin, Minocin, Solodyn, Vectrin), penicillin (BeePen-VK, Pen-Vee K, Veetids, others), tetracycline (Brodspec, Panmycin, Sumycin, Tetracap); aspirin; duloxetine (Cymbalta); fish oil (omega-3 PUFAs); pregabalin (Lyrica); alprazolam (Xanax); acetaminophen; and/or vitamins such as Vitamin B12 (cyanocobalamin); Vitamin C; Vitamin D3; Vitamin A, Vitamin E; Vitamin K, in a patient in need thereof.
64. Use of the unit dosage form as defined in any one of implementations 1 to 37 for the preparation of a medicament for reducing or eliminating drug interactions with cholestyramine, such as a blood thinner like warfarin (Coumadin, Jantoven); digoxin (digitalis, Lanoxin); propranolol (Inderal); a diuretic such as hydrochlorothiazide (HCTZ); thyroid hormones such as levothyroxine (Synthroid, Levoxyl, Levothroid); birth control pills or hormone replacement; seizure medicines such as phenytoin (Dilantin) and phenobarbital (Luminal, Solfoton); an antibiotic such as amoxicillin (Amoxil, Trimox, others), doxycycline (Adoxa, Doryx, Oracea, Vibramycin), minocycline (Dynacin, Minocin, Solodyn, Vectrin), penicillin (BeePen-VK, Pen-Vee K, Veetids, others), tetracycline (Brodspec, Panmycin, Sumycin, Tetracap); aspirin; duloxetine (Cymbalta); fish oil (omega-3 PUFAs); pregabalin (Lyrica); alprazolam (Xanax); acetaminophen; and/or vitamins such as Vitamin B12 (cyanocobalamin); Vitamin C; Vitamin D3; Vitamin A, Vitamin E; Vitamin K, in a patient in need thereof.
65. Use of the unit dosage form as defined in any one of implementations 1 to 37 for reducing or eliminating drug interactions with cholestyramine, such as estrogens, thiazide diuretics, digoxin and related alkaloids, loperamide, phenylbutazone, barbiturates, thyroid hormones, warfarin and/or some antibiotics comprising administering to a patient who is receiving concomitant administration of a medication which is known to interact with conventional orally-administered cholestyramine, in a patient in need thereof.
66. Use of the unit dosage form as defined in any one of implementations 1 to 37 for the preparation of a medicament for reducing or eliminating drug interactions with cholestyramine, such as estrogens, thiazide diuretics, digoxin and related alkaloids, loperamide, phenylbutazone, barbiturates, thyroid hormones, warfarin and/or some antibiotics comprising administering to a patient who is receiving concomitant administration of a medication which is known to interact with conventional orally-administered cholestyramine, in a patient in need thereof.
67. Use of the unit dosage form as defined in any one of implementations 1 to 37 for preventing macronutrients and/or micronutrients absorption deficiency in the gastrointestinal system of a patient in need thereof.
68. Use of the unit dosage form as defined in any one of implementations 1 to 37 for the preparation of a medicament for preventing macronutrients and/or micronutrients absorption deficiency in the gastrointestinal system of a patient in need thereof.
69. The use of implementation 68 wherein the macronutrients and the micronutrients include fat and fat-soluble vitamins.
70. The use of any one of implementations 52 to 69, wherein the daily dose of cholestyramine is 425 mg to 30 g, preferably 850 mg to 12 g, most preferably 1700 mg to 8.5 g.
71. The use of any one of implementations 52 to 69, wherein the daily dose of cholestyramine is 425 mg to 4250 mg/day, preferably 425 mg to 3400 mg/day, more preferably 425 mg/day, 850 mg/day, 1275 mg/day, 1700 mg/day, 2125 mg/day, 2550 mg/day, 2975 mg/day, 3400 mg/day, 3825 mg/day or 4250 mg/day, or ranges between any one of these amounts.
72. Unit dosage form as defined in any one of implementations 1 to 37 for treating diarrhea in a patient.
73. The unit dosage form of implementation 72, wherein the diarrhea is bile acid diarrhea (BAD).
74. The unit dosage form 72 or 73, wherein the diarrhea is chronic diarrhea.
75. The unit dosage form 72 or 73, wherein the diarrhea is associated with Bile acid malabsorption (BAM), Short bowel syndrome (SBS), ileal resection, steatorrhea (fat stool), Crohn's disease, vagotomy, diabetic vagal neuropathy, radiation and/or pruritus in patients with cholestasis.
76. The unit dosage form of implementation 75, wherein the diarrhea is associated with Short bowel syndrome (SBS),
77. The unit dosage form of implementation 72 or 73, wherein the diarrhea is associated with Type I Secondary BAM (such as ileal resection, ileal dysfunction, impaired reabsorption, e.g. Crohn's disease or necrotizing enterocolitis), Type II Primary BAM (such as IBS-D), or Type III miscellaneous associated BAM disorders (such as post-cholecystectomy, gastric surgery, chronic pancreatitis, celiac disease, SIBO, radiation, microscopic colitis).
78. Unit dosage form as defined in any one of implementations 1 to 37 for treating hypercholesterolemia, type II hyperlipoproteinemia and/or type 2 diabetes mellitus, in a patient in need thereof.
79. Unit dosage form as defined in any one of implementations 1 to 37 for treating pruritus in a patient with cholestasis.
80. Unit dosage form as defined in any one of implementations 1 to 37 for reducing or eliminating drug interactions with cholestyramine, such as a blood thinner like warfarin (Coumadin, Jantoven); digoxin (digitalis, Lanoxin); propranolol (Inderal); a diuretic such as hydrochlorothiazide (HCTZ); thyroid hormones such as levothyroxine (Synthroid, Levoxyl, Levothroid); birth control pills or hormone replacement; seizure medicines such as phenytoin (Dilantin) and phenobarbital (Luminal, Solfoton); an antibiotic such as amoxicillin (Amoxil, Trimox, others), doxycycline (Adoxa, Doryx, Oracea, Vibramycin), minocycline (Dynacin, Minocin, Solodyn, Vectrin), penicillin (BeePen-VK, Pen-Vee K, Veetids, others), tetracycline (Brodspec, Panmycin, Sumycin, Tetracap); aspirin; duloxetine (Cymbalta); fish oil (omega-3 PUFAs); pregabalin (Lyrica); alprazolam (Xanax); acetaminophen; and/or vitamins such as Vitamin B12 (cyanocobalamin); Vitamin C; Vitamin D3; Vitamin A, Vitamin E; Vitamin K, in a patient in need thereof.
81. Unit dosage form as defined in any one of implementations 1 to 37 for reducing or eliminating drug interactions with cholestyramine, such as estrogens, thiazide diuretics, digoxin and related alkaloids, loperamide, phenylbutazone, barbiturates, thyroid hormones, warfarin and/or some antibiotics comprising administering to a patient who is receiving concomitant administration of a medication which is known to interact with conventional orally-administered cholestyramine, in a patient in need thereof.
82. Unit dosage form as defined in any one of implementations 1 to 37 for preventing macronutrients and/or micronutrients absorption deficiency in the gastrointestinal system of a patient, comprising administering to a patient the unit dosage form of any one of implementations 1-37.
83. The unit dosage form of implementation 82 wherein the macronutrients and the micronutrients include fat and fat-soluble vitamins.
84. The unit dosage form of any one of implementations 72 to 83, wherein the daily dose of cholestyramine is 425 mg to 30 g, preferably 850 mg to 12 g, most preferably 1700 mg to 8.5 g.
85. The unit dosage form of any one of implementations 72 to 83, wherein the daily dose of cholestyramine is 425 mg to 4250 mg/day, preferably 425 mg to 3400 mg/day, more preferably 425 mg/day, 850 mg/day, 1275 mg/day, 1700 mg/day, 2125 mg/day, 2550 mg/day, 2975 mg/day, 3400 mg/day, 3825 mg/day or 4250 mg/day, or ranges between any one of these amounts.
86. A process for making the unit dosage form as defined in any one of implementations 1 to 37, comprising
   1) blending of cholestyramine
   2) encapsulating and
   3) enteric coating.
87. The process of implementation 86, further comprising a step of sealing the capsule before the step of enteric coating for making the unit dosage form as defined in any one of implementations 19 to 37.
88. The process of implementation 87, wherein the process is for large scale manufacture.
89. The process of implementation 87 or 88, wherein the steps of sealing the capsule and of enteric coating is comprises pan coating.

### DESCRIPTION OF THE DRAWINGS

Aspects of the invention are shown in the attached drawings, which are provided by way of non-limiting examples, wherein

FIG. 1 represents the Subject 003 gastrointestinal transit profile and time of capsule release following the oral administration of an enteric coated cholestyramine capsule (425 mg) radiolabeled with samarium-153, during the scintigraphy study.

FIG. 2 represents the Subject 005 gastrointestinal transit profile and time of capsule release following the oral administration of an enteric coated cholestyramine capsule (425 mg) radiolabeled with samarium-153, during the scintigraphy study.

FIG. 3 represents the Linear Profile of Mean Plasma Concentrations versus Time for Hydrochlorothiazide (n=17 for Treatment-A and n=18 for Treatment-B and -C)−(TPD and FDA) (Phase I drug-drug interaction (DDI) study).

FIG. 4 represents the Logarithmic Profile of the Mean plasma Concentration versus Time for Hydrochlorothiazide (n=17 for Treatment-A and n=18 for Treatment-B and -C)−(TPD and FDA) (Phase I drug-drug interaction (DDI) study).

### DETAILED DESCRIPTION

The human gastrointestinal tract consists of the esophagus, stomach, the small intestine and the colon. The small intestine is divided into three structural parts. The duodenum is the first section of the small intestine. It is about 20-25 cm long. It receives the gastric chyme from the stomach, together with digestive juices from the pancreas (digestive enzymes) and the liver (bile). The bile emulsifies fats into micelles. The stomach acids contained in gastric chyme are neutralized. The jejunum is the midsection of the small intestine, connecting the duodenum to the ileum. It is about 2.5 m long. Products of digestion (sugars, amino acids, and fatty acids) are absorbed into the bloodstream here. The ileum is the final section of the small intestine. It is about 3 m long. It absorbs mainly vitamin B12 and bile acids, as well as any other remaining nutrients. The ileum joins to the cecum of the large intestine at the ileocecal junction. The colon is the last part of the digestive system. It extracts water and salt from solid wastes before they are eliminated from the body. Unlike the small intestine, the colon does not play a major role in absorption of foods and nutrients.

The main function of bile acids is to allow digestion of dietary fats and oils by acting as a surfactant that emulsifies them into micelles. During normal digestion, bile acids are secreted into the intestines and are then re-absorbed from the intestinal tract and returned to the liver via the enterohepatic circulation. Only very small amounts of bile acids are found in normal serum. In some medical conditions, bile acids are insufficiently reabsorbed from the intestinal tract and make their way to the colon, stimulate electrolyte and water secretion, giving rise to BAD.

An increased secretion of bile acids produces an increase in bile flow. If present in excess, bile acids produce diarrhea. The current medicinal treatment of bile acid malabsorption aims at binding excess bile acids in the gastrointestinal tract, beginning in the proximal part of the small bowel, thereby reducing the secretory actions, of the bile acids. For this purpose, cholestyramine is commonly used as the bile acid sequestrant. It is an anion-exchange resin in the chloride form, consisting of styrene-divinylbenzene copolymer with quaternary ammonium functional groups.

Cholestyramine powder for oral suspension has been used worldwide for decades as an effective serum cholesterol lowering agent as well as for the treatment of bile acid diarrhea at doses between 4-24 g/day, i.e. 4 g of cholestyramine resin, one to six times daily.

Although treatment with these conventional resins or sequestrants is effective in 75-88% of patients, there are two physiological disadvantages. Firstly, the jejunal concentration of non-sequestered bile acids is below the optimum for sufficient solubilisation of the lipolytic products after meals and thus the result is malabsorption of fat, steatorrhea, and diarrhea induced by fatty acids. Secondly, malabsorption of bile acids is increased because sequestered bile acids are not available for small intestinal absorption. Moreover, the reduced reabsorption of bile acids leads to a decreased bile acids pool and synthesis. Another main concern with the use of conventional resins (sequestrants) is poor tolerance, with discontinuation rates of over 40%. Many of these effects are due to palatability or upper abdominal symptoms. Currently, the only approved formulation of cholestyramine is as a powder for oral suspension. Although it is intended to be mixed in water for drinking, it is not soluble and remains a suspension. As a result, the mixture has a texture that is described as undesirable and sandy, as well as having an unpleasant taste. Thus, many patients do not like to take it and its benefit in the treatment of BAD has been limited. In addition, conventional sequestrants may interact with other drugs as well as fat soluble vitamins. Cholestyramine resin may delay or reduce the absorption of concomitant oral medications such as thyroid and thyroxine preparations, warfarin, hydrochlorothiazide (HCTZ), beta blockers such as propranolol, phenylbutazone, phenobarbital, tetracycline, penicillin G, statins and digitalis, as well as therapeutic bile acids such as ursodiol and obeticholic acid. These medications are mostly absorbed in the proximal portion of the small intestine, such as the duodenum and the proximal jejunum. This is where a complexation with cholestyramine may occur. Thus, patients are advised to take concomitant drugs either one hour before, or 4-6 hours after, taking cholestyramine which is an inconvenience for most patients.

Therefore, there could be a therapeutic advantage to ingest a cholestyramine in a delayed release capsule form that releases the active substance in a more distal portion of the small intestine, beyond the segments involved in fat digestion and concomitant drugs absorption, thereby not influencing concomitant malabsorption of bile acids and fat, but still preventing their osmotic, laxative effects. In this context, a novel formulation that delivers cholestyramine to the lower intestine and colon may have several advantages including a more favorable patient acceptance profile, fewer drug interactions and better tolerability.

A new enteric-coated cholestyramine capsule (ECC) has been developed to manage diarrhea, more particularly diarrhea associated with SBS in patients who still have their transverse and descending colon. The new enteric-coated cholestyramine capsule can release cholestyramine in a more distal segment of the intestinal tract, downstream to the duodenum, beyond the segments involved in fat digestion and concomitant absorption, in order to bind excess bile acids before they induce diarrhea. It is also hypothesized that delivering cholestyramine in a more distal intestinal segment will prevent or reduce the magnitude of drug-drug interactions.

The new ECC capsule formulation described herein contains the same active ingredient as the already marketed powder, i.e. cholestyramine. Therefore, all previously reported pharmacological and toxicological data on this drug are relevant to the new ECC capsule formulation.

These capsules are designed to disintegrate at a pH of 6.0-7 as mirrored in the environment of the mid-jejunum to ileum, preferably in the ileum, releasing cholestyramine distally and after the stomach and the duodenum and/or distally and prior to the colon, delivering maximal small particles to sequester non-reabsorbed bile acids.

In one aspect, the UDF comprises a coated capsule intended to release the capsule contents at pH>6.0, preferably at pH>6.2. In one aspect, in vitro dissolution using a USP Type 3 apparatus meets the following specifications: pH 1.2: no disintegration of capsules for 1 hour; pH 6.2-7, preferably 6.5-6.8, more preferably 6.5 or 6.8: bursting of capsule and dispersion of the capsule contents within 30 mins. Optionally, the coated capsule may also meet the following specification: pH 6.0: bursting of capsule and dispersion of capsule contents after at least 45 mins.

In one aspect, the invention relates to a UDF in the form of a capsule, the contents of which comprise a capsule fill comprising at least 70% w/w cholestyramine, more preferably at least 75% w/w cholestyramine, more preferably at least 80% w/w cholestyramine, even more preferably at least 83% w/w cholestyramine, and most preferably at least 85% w/w cholestyramine.

The capsule coating comprises one or more enteric polymers to preferably target delivery of the capsule contents in the distal part of the small intestine, more preferably in the ileum.

The capsule may be comprised of gelatin, agar, xanthan gum, karaya gum, locust bean gum, gum arabic, pullulan, methyl cellulose, hydroxyethyl cellulose, hydroxypropyl methylcellulose, sodium alginate, and combinations thereof, preferably HPMC. Preferably the capsule is a size 0, 00 or 000 capsule, more preferably size 00.

The enteric polymer may be an acrylate or acrylic acid polymer or co-polymer, generally referred to as “acrylic polymer” hereinafter. The acrylic polymer may comprise one or more ammonio methacrylate copolymers. Ammonio methacrylate copolymers are well known in the art, and are described in NF XVII as fully polymerized copolymers of acrylic and methacrylic acid esters with a low content of quaternary ammonium groups. The acrylic polymer may be used in the form of an acrylic resin lacquer in the form of an aqueous dispersion, such as that which is commercially available from Rohm Pharma under the tradename Eudragit® or from Colorcon under the tradename Acryl-EZE®. The enteric coating may comprise a mixture of two acrylic resin lacquers commercially available from Evonik under the tradenames Eudragit® RL 30 D and Eudragit® RS 30 D, respectively. Eudragit® RL 30 D and Eudragit® RS 30 D are copolymers of acrylic and methacrylic esters with a low content of quaternary ammonium groups, the molar ratio of ammonium groups to the remaining neutral (meth)acrylic esters being 1:20 in Eudragit® RL30 D and 1:40 in Eudragit® RS 30 D.

The enteric polymer may also be a coating agent selected from the group consisting of co-polymers based on polymethacrylic acid and methacrylates, ethyl acrylate and methyl acrylate, co-polymers of acrylic and methacrylic acid esters, hydroxypropyl methylcellulose phthlate, hydroxypropyl methylcellulose acetate succinate, cellulose acetate phthalate, polyvinyl acetate phthalate or mixtures thereof. When the enteric polymer is an acrylate copolymer, it may be any pharmaceutically acceptable copolymer comprising acrylate monomers. Examples of acrylate monomers include, but are not limited to, acrylate (acrylic acid), methyl acrylate, ethyl acrylate, methacrylic acid (methacrylate), methyl methacrylate, butyl methacrylate, trimethylammonioethyl methacrylate and dimethylaminoethyl methacrylate. Several acrylate copolymers are known under the trade name Eudragit®. Another example of enteric polymer is poly(methacrylic acid-co-ethyl acrylate) 1:1, a methacrylic acid copolymer, sold under the trade name Eudragit® L 30 D. Preferably, the enteric polymer is Poly(methacrylic acid-co-ethyl acrylate) 1:1 as known as Methacrylic Acid-Ethyl Acrylate Copolymer (1:1) CAS 25212-88-8.

The capsule fill and/or the enteric coating may further comprise excipients such as bulking agents or diluents, glidants, lubricants, and other common excipients.

Suitable bulking agents or diluents include, for example, dextrose, lactose, glucose, glycine, inositol, mannitol, sorbitol, sucrose, a polyethyleneglycol (PEG), or a polyvinylpyrrolidine (PVP), or a combination thereof, preferably lactose monohydrate.

Suitable glidants include, for example, calcium phosphate, calflo E, cellulose (powder), colloidal silicon dioxide, magnesium silicate, magnesium trisilicate, silicon dioxide, starch, talcum powder, or a combination thereof, preferably colloidal silicon dioxide.

Suitable lubricants include, for example, magnesium stearate, sodium stearyl fumarate, hydrogenated castor oil, hydrogenated soybean oil, polyethylene glycol, or a combination thereof, preferably magnesium stearate.

For reasons of patient compliance, it is desirable that the total volume of the formulation is kept as low as possible. The cholestyramine content of the fill should for that reason be as high as possible. The fill of the UDF contains at least 70% w/w cholestyramine, more preferably at least 75% w/w cholestyramine, more preferably at least 80% w/w cholestyramine, even more preferably at least 83% w/w cholestyramine, and most preferably at least 85% w/w cholestyramine.

The amount of cholestyramine in the UDF preferably ranges from 350 to 500 mg, more preferably 400 to 450 mg, most preferably 425 mg.

In a preferred embodiment, the fill comprises, consists essentially of, or consists of from 80 to 90% w/w cholestyramine, from 10 to 20% w/w of a bulking agent, from 0.25 to 2% w/w of a glidant and from 0.25 to 2% w/w of a lubricant, the total of the fill being 100% w/w.

In a preferred embodiment, the amount of fill in the UDF ranges from 400 to 600 mg, more preferably 450 to 550 mg. Preferably, the fill is prepared by dry blending the ingredients, as known in the art. See, e.g., Deveswaran et al, “Concepts and Techniques of Pharmaceutical Powder Mixing Process: A Current Update,” Research J. Pharm. and Tech. 2 (2), 245-249, April-June 2009.

In a preferred embodiment, the enteric polymer coating level comprises between 5% and 15%, between 6% and 15% w/w of the UDF, preferably between 5.5% and 10%, most preferably between 5.8% and 6.8%, and even more preferably between 6.0 and 6.5%.

In order to improve the adherence of the enteric polymer onto the capsule shell, or in order to minimize the interaction between the enteric polymer and the shell, an additional barrier coating (a.k.a. seal coat) may optionally be present between the shell of the capsule and the enteric polymer coating. In a preferred embodiment, the barrier coating level comprises 6% to 15% w/w of the UDF, preferably 7% to 12%, and most preferably 9% to 11%. A particularly suitable material for the barrier coating comprises hydroxypropyl methylcellulose (HPMC).

The enteric polymer coating and/or the optional barrier coating may comprise one or more additives, such as acids and bases, plasticizers, glidants, and surfactants. Examples of suitable acids include organic acids such as citric acid, acetic acid, trifluoroacetic acid, propionic acid, succinic acid, glycolic acid, lactic acid, malic acid, tartaric acid, ascorbic acid, pamoic acid, maleic acid, hydroxymaleic acid, phenylacetic acid, glutamic acid, benzoic acid, salicylic acid, mesylic acid, esylic acid, besylic acid, sulfanilic acid, 2-acetoxybenzoic acid, fumaric acid, toluenesulfonic acid, methanesulfonic acid, ethane disulfonic acid and oxalic acid, and inorganic acids such as hydrochloric acid, hydrobromic acid, sulphuric acid, sulfamic acid, phosphoric acid and nitric acid. Examples of suitable bases include inorganic bases such as sodium bicarbonate, sodium hydroxide and ammonium hydroxide. Examples of suitable plasticizers include triethyl citrate, glyceryl triacetate, tributyl citrate, diethyl phthalate, acetyl tributyl citrate, dibutyl phthalate and dibutyl sebacate. Examples of suitable glidants include talc, glyceryl monostearate, oleic acid, medium chain triglycerides and colloidal silicon dioxide. Examples of suitable surfactants include sodium dodecyl sulfate, polysorbate 80 and sorbitan monooleate.

The coatings may be applied onto the capsule by methods known in the art, such as a fluidized bed method, a rotating fluidized bed method, a side-vented pan or coating pan method, preferably a coating pan.

Cholestyramine can affect absorption of over 300 other medications or nutrients, such as a blood thinner like warfarin (Coumadin, Jantoven); digoxin (digitalis, Lanoxin); propranolol (Inderal); a diuretic such as hydrochlorothiazide (HCTZ); thyroid hormones such as levothyroxine (Synthroid, Levoxyl, Levothroid); birth control pills or hormone replacement; seizure medicines such as phenytoin (Dilantin) and phenobarbital (Luminal, Solfoton); an antibiotic such as amoxicillin (Amoxil, Trimox, others), doxycycline (Adoxa, Doryx, Oracea, Vibramycin), minocycline (Dynacin, Minocin, Solodyn, Vectrin), penicillin (BeePen-VK, Pen-Vee K, Veetids, others), tetracycline (Brodspec, Panmycin, Sumycin, Tetracap); aspirin; duloxetine (Cymbalta); fish oil (omega-3 PUFAs); pregabalin (Lyrica); alprazolam (Xanax); acetaminophen; and/or vitamins such as Vitamin B12 (cyanocobalamin); Vitamin C; Vitamin D3; Vitamin A, Vitamin E; Vitamin K.

Another embodiment of the invention is a method for reducing or eliminating drug interactions with cholestyramine by administering a formulation according to the invention to a person who is receiving concomitant administration of a medication which is known to interact with conventional orally-administered cholestyramine. “Concomitant” as used herein refers to the administration of at least two drugs to a patient either simultaneously, sequentially, or within a time period during wherein both drugs are present or proximal in the same physiological location (e.g., stomach, duodenum, jejunum or ileum), the first administered drug has an operative effect on the second administered drug, or vice versa and/or the effects of the first administered drug are still operative in the patient.

Because of its very low solubility in aqueous environment, cholestyramine “release” refers to the availability of the cholestyramine to the intestinal content in order to bind components (i.e., bile acids) therein. Furthermore, “release” is intended to preferably refer to the beginning of the release rather than the complete release. When a capsule fill is released from the capsule, it starts with a small leak, which then broadens, before the whole capsule fill is available to the intestinal content. The capsule fill is progressively released from the capsule, due to the pH and the digestion time.

The low solubility of cholestyramine in aqueous environment may prevent the release of cholestyramine from the formulation to be measured directly. The availability of the cholestyramine to the intestinal content over time and at different pH values may instead be determined in vitro, such as by measuring the sequestering capacity of the formulation under simulated conditions for the gastrointestinal tract. Such a method may involve measuring the decreasing amount of free bile acid (i.e., the compound to be sequestered) in a liquid medium representative of the gastrointestinal tract. See also the Official Monograph for cholestyramine resin (United States Pharmacopia USP 40, NF35th edition, page 3404).

The formulations of the invention may be used as a hypolipidemic agent in the treatment of hypercholesterolemia, type II hyperlipoproteinemia and in type 2 diabetes mellitus. The formulations also may be used for the relief of diarrhea. For example, but not limitation, the diarrhea may be chronic diarrhea. For example, but not limitation, the diarrhea may be associated with SBS, ileal resection, Crohn's disease, vagotomy, diabetic vagal neuropathy and radiation, as well as for the treatment of pruritus in patients with cholestasis. The formulations may be used for diarrhea associated with Type I Secondary (ileal resection and Crohn's), Type II Primary (IBS-D), and Type III miscellaneous associated disorders (Post-cholecystectomy, gastric surgery, chronic pancreatitis, celiac disease, SIBO, radiation, microscopic colitis).

In some embodiments, daily doses of the formulations may range from 425 mg to 30 g, preferably 850 mg to 12 g, most preferably 1700 mg to 8.5 g, depending on the condition to be treated. In some embodiments, for example but not limitation treatment of diarrhea (preferably bile acid diarrhea), daily doses of the formulations may range from 425 mg to 4250 mg/day, preferably 425 mg to 3400 mg/day, more preferably 425 mg/day, 850 mg/day, 1275 mg/day, 1700 mg/day, 2125 mg/day, 2550 mg/day, 2975 mg/day, 3400 mg/day, 3825 mg/day or 4250 mg/day, or ranges between any one of these amounts.

A patient or a subject is a mammal, preferably a human, either an adult or a child. A patient usually refers to someone suffering from a disease or a condition, such as the ones described herein, whereas a subject can refer to both a healthy subject and a patient.

“Large scale manufacture” of a unit dosage form refers to manufacture for the purpose other than only lab testing. More particularly, it refers to at least I10,000, at least 50 000, at least 100 000, at least 500 000, at least 1 000 000 unit dosage forms manufactured in a same batch or lot, or a manufacturing batch or lot of at least 15 Kg, at least 20 kg, preferably at least 50 Kg, more preferably at least 100 Kg and even more preferably, at least 120 Kg of unit dosage forms.

Further, the word “example” is used herein to mean “serving as an example, instance, or illustration.” Any aspect described herein as “example” is not necessarily to be construed as preferred or advantageous over other aspects. Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C. Nothing disclosed herein is intended to be dedicated to the public regardless of whether such disclosure is explicitly recited in the claims.

It is intended that the values referred therein also refer to “about” said values, preferably about plus or minus 10%. Therefore, a variation of 1, 2, 3, 4, 5, 6, 7, 8, 9 and 10% of a value is included in the term “about”. Unless indicated otherwise, use of the term “about” before a range applies to both ends of the range. When the position or the length of sections of the small intestine are mentioned, the person of ordinary skill in the art would understand that it may vary from one subject to another, due for instance to his/her sex, age, height, etc. The same is intended for time, temperature, pH, yield values. It is also intended that when an interval of values is mentioned, although some specific values might be mentioned, all values within that interval are covered.

The invention is further illustrated by means of the following examples, which do not limit the invention in any respect. All cited documents and references are incorporated herein by reference.

### EXAMPLES

### Example 1—Cholestyramine Enteric Coated Powder

In view of the large dose of cholestyramine required for treatment and of the flexibility of using a powder for oral administration, Applicant first attempted to directly apply an enteric coating on cholestyramine powder to develop a delayed-release formulation of the powder. An enteric coating with a pH trigger of about 6.8-7 was first selected to deliver the API closer to the colon.

An important aspect in the design of an enteric coated cholestyramine powder product was particle size since the dosage form was intended to be reconstituted in a liquid vehicle for administration. To ensure patient compliance, mouth feel of the dispersed final product was very important. Therefore, granulation had to be avoided and particle size of the product was targeted to 300 μm to ensure minimum grittiness mouth feel•. A coating process was first selected to apply enteric protection polymer directly on cholestyramine API particles.

To achieve this, fluid bed equipment with bottom spray assembly was used. Initial development trials showed that it was difficult to process particles below 100 μm as they tended to be cohesive. Fine particles tended to stick (granulate) together. Similarly, a wide particle size distribution range was not suitable for powder coating, as the smaller particles tended to stick to larger ones. Therefore, the drug substance cholestyramine was sieved to use a narrower particle size distribution of 106-150 μm (sieve cut 100/140 mesh). A seal coat had to be used to prevent interaction between the enteric coating and the resin and to provide a substrate for applying the enteric polymer.

Seal Coat Selection

Two different seal coat polymers were first evaluated; HPMC (Aqueous) based seal coat and an ethylcellulose (Organic solvent) based seal coat. Both of these seal coat polymers were also evaluated with two different enteric polymers; Eudragit® FS30D and Eudragit® S 100. These enteric polymers were selected based on their ability to release at pH 7.0 and above (provides effective dissolution at pH above 7).

Eudragit® S 100F is an anionic copolymer based on methacrylic acid and methyl methacrylate (Poly(methacylic acid-co-methyl methacrylate) 1:2; CAS number: 25086-15-1). It is soluble in digestive fluids by salt formation. The product contains 0.3 Sodium Laurylsulfate (SLS). The ratio of the free carboxyl groups to the ester groups is approx. 1:2. Based on SEC method its average molar mass (Mw) is approximately 125,000 g/mol.

S30D is an anionic copolymer based on methyl acrylate, methyl methacrylate and methacrylic acid (Poly(methyl acrylate-co-methyl methacrylate-co-methacrylic acid) 7:3:1; CAS #26936-24-3). It is insoluble in acidic media, but dissolves by salt formation between pH 7.0 and pH 7.5. It is used as an aqueous dispersion with 30 dry substance. The dispersion further contains 0.3% Sodium Laurilsulfate (SLS) and 1.2% Polysorbate 80. The ratio of the free carboxyl groups to the ester groups is approx. 1:10. Based on SEC method the weight average molar mass (Mw) of the Poly(methyl acrylate-co-methyl methacrylate-co-methacrylic acid) polymer is approx. 280,000 g/mol.

TABLE 1
Comparison of initial formulation prototypes tested

| | Lot # | | | | |
|---|---|---|---|---|---|
| Ingredients | COEP-003 | COEP-006 | COEP-009 | COEP-011 | COEP-013 |
| API | Cholestyramine | Cholestyramine | Cholestyramine | Cholestyramine | Cholestyramine |
| | 100/140 | 100/140 | 100/140 | 100/140 | 100/140 |
| | mesh cut | mesh cut | mesh cut | mesh cut | mesh cut |
| Seal coating | N/A | | | | |
| ingredients | | | | | |
| Hypromellose | | 91.00% | — | 90.95% | — |
| E5LV | | | | | |
| Ethylcellulose | | — | 83.35% | — | 83.35% |
| 20cp | | | | | |
| Triethyl citrate | | 9.00% | 16.67% | 9.05% | 16.67% |
| Purified water | | X | — | X | — |
| Alcohol 95% | | — | X | — | X |
| Enteric coating | | | | | |
| ingredients | | | | | |
| Eudragit FS30D | 95.20% | — | — | 90.95% | 90.95% |
| Eudragit S100 | — | 90.86% | 90.95% | — | — |
| Triethyl citrate | 4.80% | 9.14% | — | 9.05% | 9.05% |
| Dibutyl sebacate | — | — | 9.05% | — | — |
| Purified water | X | — | — | X | X |
| Isopropyl alcohol | — | X | X | — | — |
| (90-95%) | | | | | |

All lab scale coating trials were performed using the same equipment; a mini-Glatt fluid bed, with bottom spray assembly. The API was sieved, weighed and directly added inside the product chamber. Then, the seal coat was applied and the weight gain was calculated based on the amount sprayed. The enteric coating dispersion was applied and calculated in the same manner.

TABLE 2
Manufacturing conditions tested for seal and enteric coatings.

| Lot # | COEP-003 | | COEP-006 | | COEP-009 | | COEP-011 | | COEP-013 | |
|---|---|---|---|---|---|---|---|---|---|---|
| Batch size | 80 | g | 65 | g | 65 | g | 70 | g | 70 | g |
| API | Cholestyramine | | Cholestyramine | | Cholestyramine | | Cholestyramine | | Cholestyramine | |
| | 100/140 | | 100/140 | | 100/140 | | 100/140 | | 100/140 | |
| | mesh cut | | mesh cut | | mesh cut | | mesh cut | | mesh cut | |
| Equipment | Mini-glatt | | Mini-glatt | | Mini-glatt | | Mini-glatt | | Mini-glatt | |
| Coating | | | | | | | | | | |
| parameters | | | | | | | | | | |
| Seal coating | n/a | | COEP-004 | | COEP-007 | | COEP-010 | | COEP-012 | |
| Process air | | | 0.13 | bar | 0.12-0.14 | bar | 0.11-0.12 | bar | 0.12-0.15 | bar |
| Product | | | 29-36° | C. | 26-29° | C. | 34-39° | C. | 26-30° | C. |
| temperature | | | | | | | | | | |
| Spray rate | | | 0.7 | | 0.8-1.3 | | 0.4-1.3 | | 0.5-1.2 | |
| (g/min) | | | | | | | | | | |
| Atomizing | | | 1.7-1.9 | bar | 1.7 | bar | 1.7 | bar | 1.7-1.8 | bar |
| pressure | | | | | | | | | | |
| Enteric coating | | | | | | | | | | |
| Process air | 0.11 | bar | 0.12 | bar | 0.12-0.13 | bar | 0.12-0.15 | bar | 0.11-0.15 | bar |
| Product | 26-27° | C. | 26-30° | C. | 27-29° | C. | 26-27° | C. | 25-27° | C. |
| temperature | | | | | | | | | | |
| Spray rate | 0.8-1.7 | | 0.6-1.5 | | 0.7-1.8 | | 1.0-1.5 | | 0.9-1.6 | |
| (g/min) | | | | | | | | | | |
| Atomizing | 1.6 | bar | 1.6 | bar | 1.7 | bar | 1.7-1.8 | bar | 1.7-1.8 | bar |
| pressure | | | | | | | | | | |

Furthermore, several process conditions have been tested by varying the pressure of the air in the process (0.11 to 0.15 bar), the temperature of the product (26-39C), the spray rate (0.4 to 1.8 g/min) and the atomizing pressure (1.6 to 1.8 bar). All seal coating processes were difficult to perform, due to poor fluidization of the API inside the equipment. The building up of static charges as the coating weight gain increased caused multiple nozzle blocking and powder sticking issues and thus multiple coating interruptions. For lots COEP-011 and COEP-013, a filling agent (sugar spheres) was added for enteric coating process, due to poor yield of their respective seal coating (sugar spheres were removed at the end of the coating process based on size).

Acid Resistance

Resistance to acid of the enteric coated powder was evaluated by measuring the binding capacity of EC powder with bile acid salts, in a medium that has an acidic pH (pH 4.5). The cholestyramine powder sample and bile acid salts (GCA, GDCA and TDCA) are incubated at pH 4.5 for two hours, and then the bound fraction of bile acid salts is quantified and reported as a percentage of bile acid binding. The binding test was performed at pH 4.5 because it was the lowest pH value where bile acid salts were still soluble.

TABLE 3
Bile acid binding results of lab scale manufactured product.

| | | Seal | Nature of | Enteric | Bile acid | |
|---|---|---|---|---|---|---|
| | Nature of seal | coat | enteric | coat | binding | STDev |
| Lot # | coat | (%) | coat | (%) | (%) | (Diff %) |
| API | N/A | 0 | N/A | 0 | 47.1 | 41 |
| COEP- | N/A | N/A | FS 30D | 35 | Same as | n/a |
| 003 | | | | | free API | |
| COEP- | HPMC E5 LV | 15 | S100 | 35 | 46 0 | 4.4 |
| 006 | (Aqueous) | | | | | |
| COEP- | Ethylcellulose | 15 | S100 | 35 | 42 8 | 8.5 |
| 009 | 20 cps | | | | | |
| | (organic) | | | | | |
| COEP- | FIPMC E5 LV | 15 | FS 30D | 35 | 11.8 | 4.4 |
| 011 | (Aqueous) | | | | | |
| COEP- | Ethylcellulose | 15 | FS 30D | 35 | ±11 | n/a |
| 013 | 20 cps | | | | | |
| | (organic) | | | | | |

Lot COEP-003, that did not have a seal coat, resulted in the same binding capacity as the neat API, which suggested that no acid resistance was achieved. Lots COEP-006 and COEP-009, were coated with 15% weight gain of HPMC or ethylcellulose seal coat, they were then both coated with 35% Eudragit S 100. No significant reduction in bile acid binding capacity was observed, suggesting that no acid protection of the cholestyramine was achieved using polymer Eudragit S 100. Lot COEP-011 and COEP-013 both showed a significant reduction in bile acid binding capacity at pH 4.5, which suggested that Eudragit FS30D was the better enteric polymer to pursue development trials for this product. Lot COEP-013 was selected as the formulation to further develop.

Three lots of EC powders having the same formulation as lot COPE-013 were further prepared and used to determine the effect of seal coat and enteric coat weight gains (and therefore thickness) on acid resistance. Formulations having the following seal coating (SC) and enteric coating (EC) weight gains were tested: 20% SC: 30% EC; 30% SC:20% EC and 18% SC:30% EC. However, all formulations prepared failed to show acid resistance as the bile acid binding at pH4.5 was similar as the uncoated API (data not shown).

As opposed to COPE-013, these lots had not been manufactured in the presence of a filling agent (sugar spheres) which had facilitated the flow of powder inside the fluid bed dryer. The results of COEP-011 and COEP-013 could not be reproduced by manufacturing other lots similar to COEP-013 lots without filling agent. This suggests that an adequate fluidization of the powder inside the fluid bed is important to achieve proper coating on powder particles.

In order to improve the flow of cholestyramine inside the manufacturing equipment, talc was first added in the seal coating formulation (75% w/w Ethycellulose 20 cp, 10% w/w Triethyl citrate and 15% w/w talc). Furthermore, seal coat was applied up to an 80% weight gain to further improve acid resistance. Again, this new lot (Lot COEP-030) failed to demonstrate satisfactory acid resistance (despite the use of talc and application of a thick seal coat (80% weight gain) and of an enteric coat (20% weight gain). The slight reduction in bile acid binding at pH 4.5 seemed to be attributable to the presence of excess seal coating. The product remained very difficult to process at lab scale, even after the addition of talc (very cohesive, static charge building up, etc.).

Due to limitations of the lab scale equipment, (small expansion chamber, low dew point of the lab scale equipment, air volume and atomizing air pressures limitations) it was not possible to further improve the flow of powder at this scale. It was thus decided to perform subsequent coating trials in larger equipment (Glatt GPCG-5). This equipment has a bigger expansion chamber and it does not use compressed air, therefore the dew point on the inlet air is higher.

As per lab scale recommendations, two trials were performed in larger equipment; a Glatt fluid bed GPCG-5, equipped with bottom spray Wurster assembly. Batch size of 0.8 to 1.5 kg was used in the equipment to obtain optimized fill of product bowl (based on cholestyramine's bulk density).

Formulation selected for first trials in GPCG-5 was the formulation of COEP-011.

TABLE 4
Coating formulations of two first lots performed on GPCG-5

Lot #

| Ingredients | COEP-033 | COEP-035 |
|---|---|---|
| API | Cholestyramine | Cholestyramine |
| | (not sieved) | (not sieved) |
| Seal coating ingredients | | |
| Hypromellose E5 LV | 90.95% | 90.95% |
| Triethyl Citrate | 9.05% | 9.05% |
| Purified water | 10% solid | 10% solid |
| | dispersion | dispersion |
| Enteric coating ingredients | | |
| Eudragit FS3OD | 90.95% | 90.95% |
| Triethyl Citrate | 9.05% | 9.05% |
| Purified water | 20% solid | 20% solid |
| | dispersion | dispersion |

Changing equipment from the mini-Glatt to Glatt GPCG-5 significantly improved fluidization of powder. Furthermore, sticking issue due to static charge build up was almost completely resolved. However, at higher coating levels, the powder tended to clump together when fluidization was stopped, for both seal coating and enteric coating process. This resulted in poor overall yield at the end of the two coatings (36% overall coating yield for COEP-033 and around 63% for COEP-035). At the end of coating, the material was sieved and bile acid binding of the different sieve cuts was evaluated separately.

As presented in table 5, coating trials demonstrated a lower bile acid binding capacity in acidic media (COEP-033 and COEP-035), as compared to the uncoated API (47.1%±4.1%). Results similar to COEP-011 were achieved.

TABLE 5
Binding results for first coating trials performed in Glatt GPCG-5

| | Seal | Enteric | Bile acid | STDev |
|---|---|---|---|---|
| Lot # | coat (%) | coat (%) | binding (%) | (Diff %) |
| COEP-033 | 30% | 13.83% | 13.0% | 4.3% |
| COEP-35 | 30% | 20% | 13.8% | 5.3% |
| (25/60 | | | | |
| sieve cut) | | | | |

However, the results hereunder (Table 6) suggest that smaller powder particles do not exhibit the same extent of acid resistance.

TABLE 6
Binding results for two different sieve cut of lot COEP-035

| | Seal | Enteric | Bile acid | STDev |
|---|---|---|---|---|
| Lot # | coat (%) | coat (%) | binding (%) | (Diff %) |
| COEP-035 | 30% | 20% | 13.8% | 5.3% |
| (25/60 | | | | |
| sieve cut) | | | | |
| COEP-035 | 30% | 20% | 21.7% | 9.7% |
| (60/80 | | | | |
| sieve cut) | | | | |

These results suggest that larger particles (most likely aggregates) were better coated than the smaller particles. Although it is easier to coat larger particles, presence of larger particles is undesirable in the final product. Microscopic images revealed that COEP-035 (25-60 sieve cut, larger particles) was composed of aggregates of large particle size. Also, it was possible to observe a smoother opaque surface than the uncoated API, which confirms the presence of a coating layer. The coated particles from the 60/80 sieve cut (smaller particles), showed less aggregates and the surface of these particles appeared to be more transparent. This indicated that the smaller particles were not as effectively coated.

Preliminary Stability Data

The enteric coated intermediate of lots COEP-033 and COEP-035 tended to clump together rapidly, within a few days at room temperature. This is most likely due to the tackiness of Eudragit FS30D polymer. All EC powder samples placed on stability showed clumping in the bottle. The samples had to be gently ground with a mortar and pestle before being analysed.

After 3 months stability at 40° C./75 RH, water content had increased by 35% (to 7.95% water content), which could compromise the stability of the drug product.

In view of the difficulties encountered with the above formulations, it was decided to test a new grade of API provided by Dow which was already sieved by the manufacturer to contain particle size range of 75 to 180 μm. Furthermore, the coating formulations were slightly modified to try to improve on performance.

TABLE 7
Coating formulation of lots COEP-037 and COEP-039

Lot #

| Ingredients | COEP-037 | COEP-039 |
|---|---|---|
| API | Duolite | Duolite |
| | AP143/1016 | AP143/1016 |
| Seal coating | | |
| ingredients | | |
| Hypromellose | 80.95% | 80.95% |
| Triethyl citrate | 9.10 | 9.10 |
| Talc Lo-micron | 9.95 | 9.95 |
| Purified water | X | X |
| Enteric coating | | |
| ingredients | | |
| Eudragit FS30D | 90.90 | 89.05 |
| Triethyl Citrate | 9.10 | — |
| Talc Lo-micron | — | — |
| Plasacryl T20 | — | 10.95 |
| Purified water | X | X |

TABLE 8
Coating parameters for lot COEP-037 and COEP-039

| Lot # | COEP-037 | | COEP-039 | |
|---|---|---|---|---|
| Batch size | 1.2 | kg | 0.8 | kg |
| Equipment | GPCG-5 | | GPCG-5 | |
| Equipment parameters | | | | |
| Product retention filter | Tentacle filter | | Tentacle filter (repaired) | |
| Bottom mesh | 250 | mesh | 250 | mesh |
| Bottom Plate | Type “A” plate | | Type “A” plate | |
| Partition height (clearance) | 10 | mm | 10 | mm |
| Dew point (° C.) | +8 to +11 | | +8 to +12 | |
| Coating parameters | | | | |
| Seal coating | | | | |
| Process air | 40 | cfm | 40 | cfm |
| Product temperature | 40-41° | C. | 40-43° | C. |
| Spray rate (g/min) | 2.7 to 7.7 | | 2.7 to 7.5 | |
| Atomizing pressure | 3.0 | bar | 3.2 | bar |
| Enteric coating | | | | |
| Process air | 40 | cfm | 30 | cfm |
| Product temperature | 24-25° | C. | 25-26° | C. |
| Spray rate (g/min) | 2.7 to 8.6 | | 2.7 to 5.5 | |
| Atomizing pressure | 3.0 | bar | 3.0 | bar |

Addition of talc in the coating formulation improved the processability of the seal coating for both lots COEP-037 and COEP-039. Furthermore, the narrower particle size distribution grade also improved the coating process and the quality outcome of the coated product. Less uncoated fines were obtained and fewer aggregates were formed. As a result, no issues were encountered, and the process did not have to be stopped during seal coating of both lots. At the end of the seal coatings, the batches were sieved and only particles <250 μm were used for enteric coating. Moreover, for COEP-037, the yield after 20% weight gain of seal coat was 95%. From this amount, 92.2% of the particles were <250 μm and only 7.8% of the particles harvested were between 250-600 μm. These results suggested that very little aggregation and sticking occurred during seal coating.

The processing of the enteric coating formulation could not be enhanced with the addition of 9.95% of talc. Immediate clogging of the nozzle occurred. Therefore, COEP-037 was coated using the same enteric formulation as COEP-033 and COEP-035. Triethyl citrate was replaced with Plasacryl T20 in formulation COEP-039.

Enteric coating of COEP-037 was difficult, since the particles stuck together fairly rapidly. As soon as the fluidization was interrupted, it was impossible to resume the coating since all the powder clumped at the bottom of the bowl. Colloidal silicon dioxide had to be blended with the in-process coated particles to improve the flowability of the powder and be able to resume coating. This issue was not seen when Plasacryl (containing glyceryl monostearate, TEC and polysorbate 80) was used in lot COEP-039. Plasacryl facilitated fluidization and the flowability of the seal coated APL Furthermore, no nozzle clogging occurred with the enteric coating formulation of COEP-039.

The enteric coated intermediate of lot COEP-03 7 had a poor physical stability and the powder clumped at room temperature only after few days. Evidently addition of 0.5% colloidal silicon dioxide with the final coated particles did not prevent the issue.

After enteric coating, COEP-039 was also blended with 0.5% CSD to prevent any potential clumping of the enteric coated intermediate during storage. No clumping on storage at room temperature was observed.

As expected, higher the enteric coating weight gain, lesser the bile acid binding observed at pH 4.5. Lot COEP-039 tends to show higher binding capacity than COEP-037, for a same weight gain percentage. This could be attributed to the presence of Plasacryl T20 in the formulation of COEP-039. The differences observed in acid resistance between COEP-037 and COEP-039 could also be attributed to their particle size. With smaller particle size, there's a surface area increase. Therefore, more coating is required to cover the surface of smaller particles as compared to larger ones. However, some acid resistance was successfully achieved with lot COEP-039 (from 25% weight gain).

TABLE 9
bile acid binding results of COEP-037 and COEP-039

| | Seal | Enteric | Bile acid | STDev |
|---|---|---|---|---|
| Lot # | coat (%) | coat (%) | binding (%) | (Diff %) |
| COEP-037A | 20% | 10.0% | 45.5% | 9.9% |
| (10% enteric coat) | | | | |
| COEP-037B | 20% | 19.0% | 24.3% | 19.1% |
| (19% enteric coat) | | | | |
| COEP-037C | 20% | 25.8% | 9.1% | 12.7% |
| (25.8% enteric coat) | | | | |
| COEP-037 | 20% | 25.8% | 7.7% | 24.2% |
| (25.8% EC w/CSD) | | | | |
| COEP-039C | 20% | 20.7% | 39.7% | 20.4% |
| (20.7% enteric coat) | | | | |
| COEP-039D | 20% | 25.0% | 22.7% | 31.1% |
| (25% enteric coat) | | | | |
| COEP-039E | 20% | 30.0% | 14.8% | 14.5% |
| (30% enteric coat) | | | | |

Stability Data

The enteric coated intermediate of lot COEP-039 was selected to be placed on a limited stability study. Bottles of 40 cc HDPE, high thickness, were filled with approximately 7 grams of coated powder (COEP-039+0.5% CSD). HDPE caps were used, and all bottles were induction sealed. The coated powder was not mixed with final flavoring and dispersing excipients.

At room temperature, the material did lump together, but it was very easily recovered after gently sifting the powder (clumps formed were not hard). However, samples placed at 40° C./75 RH after 1 month could not be recuperated, the powder had completely stuck together. This was not observed for samples placed at 25° C./60 RH. Clumping at higher temperature could have resulted due to the low glass transition temperature of the Eudragit FS30D polymer (about 40° C.).

After 1 month at 40° C./75 RH, the sample had to be ground with a mortar and pestle to be able to use it. Grinding of the sample could have damaged the coating and compromised the acid resistance of the product. This could explain the slight decrease in acid resistance after 1 month of stability.

Applicants has also tested direct application of the enteric coating on the pre-sieved API using Eudragit FS30D or an alternative enteric coating (HPMCAS and fugitive salt) specially formulated to dissolve at pH 6.8-7. The objective of these experiments was to determine if acid resistance could be obtained by directly applying it on the resin. However, no acid protection was observed with either coatings and static charge build up was observed with the HPMCAS coating. Reducing product temperature and increasing spray rate resulted in large agglomerations.

Although some success had been achieved with the COEP-039 formulation and despite several improvement trials, Applicant was unable to successfully scale up the manufacturing process to an acceptable level. Agglomeration occurred in all trials performed. The process often had to be stopped because of particle agglomeration (oversizing) further requiring sieving of the batches and leading to poor yield (about 30%) and poor-quality product. In view of these difficulties, the project was abandoned.

### Example 2—Enteric Coated Minitabs

After the failed attempt to successfully develop an enteric coated powder formulation suitable for efficient large-scale manufacturing, it was decided to focus on the preparation of enteric coated minitabs. Minitabs were expected to provide the advantage of allowing a more uniform dispersion of cholestyramine at its target site. Another expected advantage of using minitabs was that they would reach their target site faster and in a more timely (constant) manner. Indeed, it was thought that their residence time in the stomach would be less affected by its content (fasting/empty state vs non-fasting state). Minitabs released in the stomach would be less subject to float at the top and more effectively sink at the base, thereby reaching their target site faster.

Three different minitabs formulations were prepared (average weight of 8 mg).

Each formulation contained:
Cholestyramine resin (between 75% and 80% w/w)
Lactose as a diluent/filler (14% w/w);
Colloidal silicone dioxide (0.48% w/w) as a glidant;
Magnesium stearate (0.5% w/w) as a lubricant; and
As a binder either:
   Povidone (2%)
   HPC SSL (10%)
   HPC SSL superfine (7%).

TABLE 10
Formulation composition

| | Lot # | | |
|---|---|---|---|
| Ingredients (% w/w) | IDDS-010* | IDDS-014 | IDDS-015 |
| Cholestyramine Resin | 80.65 | 77.93 | 75.02 |
| Lactose Flowlac 100 | 14.00 | 14.00 | 14.00 |
| Colloidal silicon dioxide | 0.48 | 0.48 | 0.48 |
| Povidone K30 | 2.20 | n/a | n/a |
| HPC SSL Regular | n/a | n/a | 10.00 |
| HPC SSL Super fine | n/a | 7.09 | n/a |
| (SFP) | | | |
| Magnesium stearate | 0.50 | 0.50 | 0.50 |

It was found that povidone was not suited as a binder. Minitabs prepared with this binder did not provide sufficient hardness and were too friable even at compression forces of 19 kN, tablets could be easily crushed by hand (hardness value of 0.70 Kp). Although, the change to HPC SSL and HPC SSL superfine significantly improved tablet hardness (to about 2 Kp), the shape of the minitablets (cylinders of 2.0×3.0 mm) was less than suitable for fluid bed coating. The long length of tablets tended to cause twining issues during coating. The blend was not easily compressible as the tablet height did not change much with increasing compression force. High variability in weight and hardness was also observed with some minitabs and feeder speed had to be increased to reduce variability issues during manufacture.

Despite the above drawbacks, different seal coatings were tested for the minitabs.

Seal Coats

All coating trials were performed on Mini-glatt equipment, using bottom spray assembly.

TABLE 11

| Coat no. 1 | Coat no. 2 | Coat no. 3 | Coat no. 4 |
|---|---|---|---|
| (batch size | (batch size | (Batch size | (Batch size: |
| 200 g, on | 150 g, on | 177 g, on | 150 g, on |
| IDDS-010) | IDSS-014) | IDDS-015) | IDSS-015) |
| HPMC E5 | HPMC E5 LV | HPMC E5 LV | HPC-L (83.35%) |
| (90.91%) | (83.3%) | (71.43%) | |
| TEC (9.09%) | TEC (8.33%) | TEC (7.14%) | Colloidal silicon |
| | | | dioxide (16.67%) |
| N.A. | Talc (8.33%) | Talc (21.43%) | N.A. |
| Target weight | Target weight | Target weight | Target weight |
| gain 10% | gain 10% | gain 10% | gain 10% |
| Dispersion 10% | Dispersion 8% | Dispersion 8% | Dispersion 9% |

All coating trials were performed on Mini-glatt equipment, using bottom spray assembly.

Seal coat No. 1: Sticking in the partition column and minitablet twining was observed. Increasing dryer conditions (from 40° C. to 50° C.) and lowering spray rate did not help (process air increased from 0.25 bar to 0.4 bar and spray rate reduced). Every 50 g of coating sprayed, the coating had to stopped to clean partition column. Minitabs were highly sticky. Coating had to be interrupted many times.

Seal coat no. 2: talc was added to reduce the sticking issue. After 3% weight gain, coating had to be stop due to the presence of more than 40% twins. Coating solution was diluted and more talc was added, the spray rate was reduced from 0.9 g/min to 0.7 g/min and product temperature increased to 48° C. Despite these adjustments, sticking of the minitablets was still observed at an unacceptable level. Coating was stopped and product discarded.

Seal coat no. 3: Addition of even more talc as compared to seal coat no. 3 did not help. Spraying of small amount of solution caused partition column to block due to sticking of minitablets. A 7.9% of weight gain was difficult to achieve. Multiple interruptions and cleaning steps were required.

Seal coat no. 4: No twins were observed. However, minitabs sticking on the column still occurred. Situation may at least be due to tablet shape. An 8% seal coat was the best that could be achieved.

Enteric Coatings:

TABLE 12
The following enteric coatings were evaluated:

| Enteric coating (EC) # 1 (% w/w) | Enteric coating (EC) # 2 (% w/w) |
|---|---|
| Eudragit ™ FS30D (30%): 91.09% | HPMCAS XCS47163 (62.11%) |
| W/W | Hypromellose Acetate Succinate |
| Poly(methyl acrylate-co-methyl | |
| methacrylate-co-methacrylic acid) | |
| 7:3:1, CAS no. 26936-24-3 | |
| Plasacryl ™ HTP20 (20%): 9.09% | TEC (17.39%) |
| | Talc (18.63%) |
| | SLS (1.86%) |
| Target weight gain 25% | Target weight gain 30% |
| % dispersion 20% | % dispersion 10% |
| Batch size: 200 g | Batch size: 150 g |

The enteric coating was performed with either Eudragit FS30D coating (EC #1, pH trigger of about 7), or HPMCAS enteric coating, which has a pH trigger of about 6. No twinning was observed with both enteric coatings. However, sticking in partition column was still an issue, especially with enteric coating #2.

The HPMCAS (aqueous dispersion) resulted in a visually good quality product.

The coating was uniform, very smooth and no visual defects were found. This HPMCAS aqueous dispersion was applied on top of seal coated mini-tablets of cholestyramine. The seal coating protected the cores from water exposition.

Observations

The cylindrical shape of the minitablets (2.0×3.0 mm) renders them fragile in the middle (they break more easily during fluidizing) and makes them difficult to coat.

Coating of cholestyramine mini tablets is difficult by fluid bed coating (bottom spray).

From previous experience with cholestyramine 500 mg, it was found that aqueous coating interacts with the surface of the tablets. This is believed to be due to the high swelling capacity of the resin in presence of water.

It was thought at first that using better drying capacity instrument (the fluid bed, bottom spray equipment) would render it possible to coat cholestyramine tablets with aqueous dispersions. However, this was proven insufficient. Indeed, from the seal coating trials, it was found under the magnifying glass that most of the tablets had a very rough surface after coating. Some layers of coating were also peeling, with powder coming off of the tablets.

Seal coating performed with an alcoholic dispersion resulted in a better adherence of the polymer film on the tablets. The film is smoother and more homogeneous. Less powder and tablet residues were found in the wurster after the coating trial, as compared to aqueous seal coating.

Aqueous coating dispersion of the enteric coating could be applied on seal coated minitablets as the presence of the seal coat protected the core from water exposition.

Acid Resistance

Minitablets coated with 8% HPC seal (SC #4) coat+25% HPMCAS coat, Lot COEP-050, were filled into gelatin capsules. Size 00 capsules were filled with 50 mini-tablets (maximum that could fit, equivalent to approx 355 mg of cholestyramine powder). 500 mL of 0.1 N HCl was added in a dissolution beaker. The capsules were put in baskets and rotated at 75 rpm.

TABLE 13

| Time | | |
|---|---|---|
| (min) | Assay #1 | Assay #2 |
| 15 | Gelatin capsule shells are | Gelatin capsule shells |
| | completely dissolve. | are completely dissolve. |
| | Dissolution media is already | Dissolution media is |
| | hazy (could be related to | already hazy (could be |
| | dissolution of capsules | related to dissolution |
| | or disintegrating minitabs) | of capsules or |
| | | disintegrating minitabs) |
| 30 | Same as above (at 15 min) | Same as above (at 15 min) |
| 60 | Removal of basket: 27/50 | Dissolution media is hazy |
| | minitabs are found intact. | |
| | Some minitabs shells found | |
| | empty with little holes by | |
| | which powder leaked | |
| 120 | N/A | Removal of basket: 25 min |
| | | tabs are found intact. Few |
| | | coting shells found empty |
| | | with some wholes by which |
| | | content leaked. |

There were no significant differences between coated minitablets exposed to acidic medium after 1 or 2 hours.

Even after 15 mins of acid exposure, the dissolution medium was found to be very hazy. It is thought that the defective minitablets (which represented about half of the total number of minitabs in a capsules) broke and dispersed rapidly, while the others showed acid resistance. After the first 15 mins, the dissolution medium showed some particles in suspension, more likely corresponding to dispersed cholestyramine. The cholestyramine powder is insoluble and had the same appearance when dispersed in water. The release of capsule content was not pH or time-dependant, but more related to the film's integrity. Approximately 10% of the mini-tablets had sharp edges (a collar), which may have compromised the integrity of the functional coating. The coating level might have been insufficient to cover these edges. Furthermore, during these first trials, a lot of sticking issues and sieving steps might have damaged the functional coating, which could explain the endpoint of the dissolution results.

Conclusions:

Uniform coating was difficult to achieve due to shape and fragility of the minitabs. This resulted in variable (uneven) quality of product and unacceptable acid resistance. Project was abandoned in view of the foregoing difficulties.

### Example 3—Development Summary of Enteric Coated Capsules

As noted above, stable, high quality, enteric coated capsules are difficult to make especially at large scale due to the difficulty in obtaining even (uniform) coatings on all capsules, particularly near the seam where the two parts of the shell are joined. Furthermore, large scale manufacturing processes and manipulations may damage the fragile enteric coating on the capsule and affect the quality of the final product and release of the active ingredient(s). Accordingly, there are very few drugs on the market with enteric coated capsules. Generally, the content of the capsule (minitabs or pellets) are coated, not the capsule itself. In such case the capsule shell helps to protect the enteric coating. Furthermore, as shown above, due to its intrinsic nature, cholestyramine powder tends to agglomerate, interact with negatively charged enteric coatings and create static on manufacturing equipment, which further complicates the manufacturing process. Nonetheless, given the difficulties encountered with the development of the enteric coated powder and minitabs, it was next decided to develop an enteric coated capsule filled with cholestyramine powder having the in vitro release/bursting profile set forth in Table 14 below.

The following disintegration time profile was established and targeted to be achieved using a pan coating process on capsules shells.

TABLE 14
target disintegration profile of cholestyramine drug product

| | Target disintegration time |
|---|---|
| Criteria | (for information) |
| Apparatus 3, pH 1.2 | 60 mins resist (no burst) |
| Apparatus 3, pH 6.0 | NLT 45 mins (burst time) |
| Apparatus 3, pH 6.8 | NMT 30 mins (burst time) |

As shown below, first, different blends of enteric coatings as well as novel blends, specifically designed to disintegrate around pH 6.5, were tested. None were proven satisfactory either because they did not enable to obtain the proper disintegration profile or were difficult to make (poor manufacturing yield). Unexpectedly, applicants were able to obtain the desired bursting profile by selecting the right amount of an anionic coating (e.g., Poly(methacrylic acid-co-ethyl acrylate, 1:1 (Eudragit L30-D55)) having a pH trigger well below the targeted pH of 6.8 (trigger pH of about 5.5); in combination with a seal coat. Although HPMC capsules are recognized as having a rough surface and to enable direct coating (as opposed to gelatin capsules), it is thought that the enteric coating did not efficiently adhere to the surface of the capsule, causing insufficient or inconsistent acid resistance and/or variable release profiles. It was further found that the use of a seal coat could significantly reduce variations in bursting profile by acting as a substrate for the enteric coating. The seal coat may also further reduce unwanted electrostatic interactions between cholestyramine and the enteric coating at the seam of the capsule.

By selecting the right combination of reagents for each of the seal coat and enteric coat, it was possible to develop a stable formulation, suitable for large scale preparation, having the desired release profile and which was shown to significantly reduce interactions between cholestyramine and other drugs, particularly those absorbed in the proximal part of the small intestine.

The new capsule described therein is obtained by the following process:

1) Blending

2) Encapsulation

3) Sealing

4) Coating.

Each of these steps has been the subject of the following experiments.

1) Blending of Cholestyramine
A 50 Kg blend (COEP-046) from commercial cholestyramine was prepared to enable preparation of a large batch of capsules for multiple coating trials.

TABLE 15
formulation of cholestyramine blend (COEP-046)

| Ingredient | % w/w | Quantity (g) |
|---|---|---|
| Cholestyramine | 88.24 | 44,120 |
| Lactose monohydrate | 10.78 | 5,390 |
| supertablet 11SD EP | | |
| Colloidal silicon dioxide | 0.48 | 240 |
| Magnesium stearate | 0.50 | 250 |
| Total | | 50,000 |

2) Encapsulation of Cholestyramine
Lot COEP-046 was encapsulated (Lot COEP-047) for a theoretical yield of 133,333 HPMC capsules (size “0”), using the automated Bosch GKF400.

2.1 Target Disintegration Time Profile

The following disintegration time profile was established for information only and was targeted to be achieved using a pan coating process on capsules shells.

TABLE 16
target disintegration profile of cholestyramine drug product

| | Target Disintegration Time |
|---|---|
| Criteria | (for information) |
| Apparatus 3, pH 1.2 | 60 mins resist (no burst) |
| Apparatus 3, pH 6.0 | NLT* 45 mins (burst time) |
| Apparatus 3, pH 6.8 | NMT** 30 mins (burst time) |

*No less than (NLT)
**No more than (NMT)

3) Sealing and Coating of the Capsule

3.1 Lot IDDS-013

HPMC capsules of size “0” were manually filled using the following formulation:

TABLE 17
formulation of blend used for IDDS-013

| Ingredient | % w/w | Quantity (g) |
|---|---|---|
| Cholestyramine | 85.02 | 425.0 |
| Lactose Flowlac 100 | 14.00 | 70.0 |
| Colloidal silicon dioxide | 0.48 | 2.4 |
| Magnesium stearate | 0.50 | 2.5 |
| Total | | 499.9 |

Roughly 500 capsules of size “0” were prepared with an average fill weight of 374.4 mg/capsule. Before coating, capsules were visually inspected, and deformed/broken capsules were removed.

IDDS-013 seal coating formulation (based on seal coating formulation used for cholestyramine EC powder):

TABLE 18
Formulation of seal coating dispersion (IDDS-013)

| Ingredients for seal coat | % w/w |
|---|---|
| HPMC E5 LV | 83.33 |
| TEC | 8.33 |
| Talc | 8.33 |
| Total | 100 |

10% solid dispersion prepared

TABLE 19
Formulation of enteric coating dispersion (IDDS-013)

| Ingredients for enteric coat | % w/w |
|---|---|
| HPMCAS JZ160-122 | 95.0 |
| Triethyl citrate | 5.0 |
| Total | 100 |

10.5% solid dispersion was prepared

The enteric polymer JZ160122 and a suggested coating formulation/preparation method were provided by Dow Chemical. HPMCAS JZ160122 is a special blend of fugitive salts and HPMCAS (neutralized), ready to disperse. That grade of polymer has a pH trigger of approximately 6.2. A direct dispersion of 10.5% solids was prepared in purified water. The dispersion was impossible to screen and spray, as it appeared to be too viscous and granular (after 1.5 hours mixing). The dispersion concentration was reduced from 10.5 to 8% solid content, and it was further mixed for 1 hour. Frequent nozzle clogging occurred, and the granular texture was still present. After almost 48 hours of mixing, the texture did not improve, with granules still visible in the dispersion. As per Dow recommendation, the solution was cooled (bath with water and ice) and further mixed for 1 hour. The dispersion became clear and homogeneous. Therefore, the method of preparation was concluded to require constant cooling of the coating dispersion. The enteric coating dispersion was kept on ice throughout the coating process.

Coating parameters, formulations and coating levels need to be optimized to achieve homogeneous coating that gives targeted disintegration time at different pH. Challenge of the coating is at the seam and on the extremities of the capsules.

TABLE 20
Coating parameters of lot IDDS-013
seal coating and enteric coating

| | Seal | Enteric |
|---|---|---|
| Parameters | (IDDS-013 seal) | (IDDS-013 enteric) |
| Batch size (g) | 200 | 200 |
| Pan size (inches) | 10″ | 10″ |
| Pan speed (rpm) | 16-17 | 16-17 |
| Inlet temp (Actual ° C.) | 47-48 | 46-49 |
| Exhaust temp (° C.) | 38-40 | 39-41 |
| Spray rate (g/min) | 2.84 | 2.8-5.6 |
| Inlet air volume (cfm) | 180-181 | 185-190 |
| Atomizing air pressure (psi) | 12.4-12.8 | 12.9-13.6 |
| Air pattern pressure (psi) | 12.6-12.7 | 13.2-13.7 |
| Weight gain (%) | 5.9% | 25.5% |

3.2 Lot COEP-048

The same seal coating formulation as IDDS-013 was used. The capsules of COEP-047 were coated as follows.

TABLE 21
Seal coating formulation of COEP-048

| Ingredients for seal coat | % w/w |
|---|---|
| HPMC E5 LV | 83.33 |
| TEC | 8.33 |
| Talc | 8.33 |
| Total | 100 |

10% solid dispersion was prepared for seal coating

Given the issues seen with the preparation and use of a new polymer grade from Dow was used HPMCAS XCS47163. This is a non-neutralized polymer formula, which has the same pH trigger of 6.2.

TABLE 22
Enteric coating formulation of COEP-048

| Ingredients for enteric coat | % w/w |
|---|---|
| HPMCAS XCS47163 | 62.11 |
| Triethyl citrate (TEC) | 17.39 |
| Talc | 18.63 |
| Sodium lauryl sulfate (SLS) | 1.86 |
| Total | 100 |

9% solid dispersion was made for the enteric coating.

The enteric coating dispersion was prepared based on the supplier's protocol for that particular enteric polymer formula. Talc was first homogenized for 15 mins, and then put under agitation with an impeller mixer. The TEC and SLS were added. After 15 mins, the HPMCAS was added and was stirred for 1 hour. This coating dispersion resulted in a very low viscosity milky-white suspension that was easy to spray.

TABLE 23
Coating parameters of COEP-048 seal coating and enteric coating

| | Seal | Enteric |
|---|---|---|
| Parameters | (COEP-048SC) | (COEP-048EC) |
| Batch size (g) | 1100 | 700 |
| Pan size (inches) | 15 | 15 |
| Pan speed (rpm) | 10-15 | 11.1 |
| Inlet temp (Actual ° C.) | 58-62 | 53-64 |
| Exhaust temp (° C.) | 37-45 | 34.3-44.6 |
| Spray rate (g/min) | 2.0-12.1 | 2.9-17.5 |
| Inlet air volume (cfm) | 202-203 | 172-216 |
| Atomizing air pressure (psi) | 19.9-25.0 | 19.8-20.1 |
| Air pattern pressure (psi) | 24.9-25.1 | 20.0-20.2 |
| Weight gain (%) | 5.9% | 35.75% |

TABLE 24
Seal coating formulation of COEP-049

| Ingredients for seal coat (COEP-049) | % w/w |
|---|---|
| Opadry 03K19229 | 80.00 |
| Talc | 20.00 |
| Total | 100 |

A 10% solid dispersion was prepared

TABLE 25
Enteric coating formulation of COEP-051 (and COEP-049)

| Ingredients for enteric coat (COEP-051) | % w/w |
|---|---|
| Eudragit FS30D (dissolution pH between 7 | 51.20 |
| and 7.5) | |
| Eudragit L30D55 (dissolution pH between | 12.80 |
| 5.5 and 6.3) | |
| Triethyl citrate | 4.00 |
| Talc | 32.00 |
| Total | 100 |

A 20% dispersion was made for the enteric coating.

Foaming of the enteric suspension (COEP-049 Enteric) was observed. Therefore, the enteric suspension was not sprayed.

Lot COEP-051 enteric coating was then prepared, where the suspension was prepared by homogenizing the talc without TEC. That resolved the foaming issue and lot COEP-051 was coated using lot COEP-049 seal coated capsules. For lot COEP-051 a mixture of two enteric coatings with different target pH were used. FS30D is described in more details above and consists of an anionic copolymer based on methyl acrylate, methyl methacrylate and methacrylic acid (Poly(methyl acrylate-co-methyl methacrylate-co-methacrylic acid) 7:3:1. L30D55 consists of an anionic polymer with methacrylic acid as a functional group (Poly(methacrylic acid-co-ethyl acrylate) 1:1 also known as Methacrylic Acid-Ethyl Acrylate Copolymer (1:1) (CAS #25212-88-8). It is provided as a 30% dispersion which further contains 0.7% Sodium Lauril sulfate and 2.3% Polysorbate 80 on solid substance, as emulsifiers. Based on SEC method the weight average molar mass (Mw) of Methacrylic Acid-Ethyl Acrylate Copolymer (1:1) is approx. 320,000 g/mol.

TABLE 26
Coating parameters of COEP-051 seal coating and enteric coating

| Parameters | Seal (COEP-049) | Enteric (COEP-051) |
|---|---|---|
| Batch size (g) | 765 | 700 |
| Pan size (inches) | 15 | 15 |
| Pan speed (rpm) | 11.1-11.2 | 11.0-11.2 |
| Inlet temp (Actual ° C.) | 52.5-65 | 28.0-35.1 |
| Exhaust temp (° C.) | 41.1-45.7 | 24.5-31.0 |
| Spray rate (g/min) | 3.2-9.1 | 4.1-8.4 |
| Inlet air volume (cfm) | 190-207 | 197-234 |
| Atomizing air pressure (psi) | 19.8-20.1 | 17.8-18.3 |
| Air pattern pressure (psi) | 24.9-25.0 | 17.9-18.1 |
| Weight gain | 5.22% | 35.95% |

Lower pan speed was used, to allow capsules to stay longer in front of spray pattern and thus to cover more homogeneously at the seam of the capsules.

The capsules coated to 10.58% and 15.64% w/w enteric coating were tested for burst time in pH 6.2 and pH 6.8, using USP Apparatus III at 10 dpm. However, the burst times were long at 70-85 mins. To note, these capsules were not exposed to the acid stage before being dipped in buffer.

3.4. Manufacture of Lot COEP-053

The ratio of the two enteric coatings was changed in an effort to obtain a shorter disintegration time (28:70 L30D55: FS30D—Table 28). The triethyl citrate was removed from the enteric formulation and was replaced by Plasacryl T20. Plasacryl also acts as an anti-tacking agent; therefore, talc was also removed from the enteric coating formulation. The seal coating dispersion was slightly modified from lot COEP-048; More talc was added in order for the talc to be used as a filler of gaps (for example at the seam) to achieve uniform coating and acid resistance of coated capsules.

TABLE 27
Seal coating formulation of COEP-053

| Ingredients for seal coat (COEP-053) | % w/w |
|---|---|
| HPMC E5 LV | 71.43 |
| Triethyl citrate | 7.14 |
| Talc | 21.43 |
| Total | 100 |

9% solid dispersion was prepared

TABLE 28
Enteric coating formulation of COEP-053

| Ingredients for enteric coat (COEP-053) | % w/w |
|---|---|
| Eudragit FS30D | 70.16 |
| Eudragit L30D55 | 28.06 |
| Plasacryl T20 | 1.77 |
| Total | 100 |

14% solid dispersion was prepared

TABLE 29
Coating parameters of COEP-053 seal coating and enteric coating

| Parameters | Seal (COEP-053) | Enteric (COEP-053) |
|---|---|---|
| Batch size (g) | 800 | 750 |
| Pan size (inches) | 15 | 15 |
| Pan speed (rpm) | 11 | 11.0-11.2 |
| Inlet temp (Actual ° C.) | 52-58 | 32.7-36.3 |
| Exhaust temp (° C.) | 41.1-42.0 | 26.1-27.8 |
| Spray rate (g/min) | 4.75-9.50 | 7.2-14.0 |
| Inlet air volume (cfm) | 204-207 | 205-215 |
| Atomizing air pressure (psi) | 20.8-21.0 | 17.9-22.0 |
| Air pattern pressure (psi) | 25.1-25.2 | 18.2-22.0 |
| Weight gain | 5.44% | 35.77% |

The texture of the enteric coating was not smooth, which could be due to a lack of plasticizer. Capsules from lot COEP-053 with coating of ˜6% showed a burst time at pH 6.2 and 6.5 of about 30 mins and 20 mins respectively. Tested capsules were not subjected to an acid test before exposure to the two pH buffers. Higher coating percentage (10.7% and more) showed disintegration time of more than 40 mins for both pH 6.2 and pH 6.5 tests (Apparatus III, 10 dpm). The disintegration time of capsules with higher coating percentages were much longer than the intended target.

3.5. Manufacture of Lot COEP-054

For lot COEP-054, the seal coated lot COEP-052 was used to apply an enteric coating. The amount of plasticizer was increased in COEP-054 coating formulation and talc was incorporated to help with the appearance of the coating. The proportion of L30D55 was increased to 30% to further reduce burst time at pH 6.2 and 6.5.

TABLE 30
Seal coating formulation of COEP-052

| Ingredients for seal coat (COEP-052) | % w/w |
|---|---|
| HPMC E5 LV | 71.43 |
| Triethyl citrate | 7.14 |
| Talc | 21.43 |
| Total | 100 |

9% solid dispersion was prepared

TABLE 31
Enteric coating formulation of COEP-054

| Ingredients for enteric coat (COEP-054) | % w/w |
|---|---|
| Eudragit FS30D | 57.9 |
| Eudragit L30D55 | 23.1 |
| Plasacryl T20 | 6.7 |
| Talc | 12.2 |
| Total | 100 |

11.8% solid dispersion was prepared

TABLE 32
Coating parameters of COEP-054 seal coating and enteric coating

| Parameters | Seal (COEP-052) | Enteric (COEP-054) |
|---|---|---|
| Batch size (g) | 800 | 750 |
| Pan size (inches) | 15 | 15 |
| Pan speed (rpm) | 14.1-16.3 | 11.0-12.2 |
| Inlet temp (Actual ° C.) | 54.0-64.3 | 31.2-33.2 |
| Exhaust temp (° C.) | 41.0-45.8 | 25.0-27.8 |
| Spray rate (g/min) | 5.7-13.2 | 4.6-13.0 |
| Inlet air volume (cfm) | 204-208 | 204-211 |
| Atomizing air pressure (psi) | 19.8-20.2 | 19.4-19.9 |
| Air pattern pressure (psi) | 25.0-25.2 | 21.5-22.0 |
| Weight gain | 10.0% | 19.91% |

The appearance of the capsules was smooth. Testing for disintegration time (Apparatus III, 10 dpm) was changed to include a prior 2-hour acid soak (0.1N HCl), followed by a change of buffer media for either pH 6.0, 6.2 or 6.5. The capsules coated to 10.15% and 15.34% coatings were found to be resistant to acid for 2-hours, when samples of 7.9% coating and less failed to resist acid stage. The samples at 10.15% and 15.34% showed a burst time of 30-90 mins in pH 6.0 and burst time of 20-30 mins in pH 6.2 and 6.5 (no difference between the two buffer media). Since the first acid exposure impacts the disintegration time obtain after buffer exposition, it was decided to use the change over method for subsequent testing. Two other testing were performed to evaluate the best method; three-stage disintegration and another two-stage disintegration method, with only 1 hour acid soaking.

For the three-stage resistance study, the capsules of COEP-054 were exposed to acid stage for 1 hour, if resistance was observed for an hour, the capsules were subjected to a buffer of pH 6.0 (1 additional hour) and finally burst time in pH 6.5 was measured. Capsules of 12.52% and 15.35% coating were tested. However, the capsules tested resisted acid stage, pH 6.0 stage and did not disintegrate within 30 mins at pH 6.5. The burst time was concluded to be too long.

For the two-stage study (1 hour acid, then pH 6.5), the samples of 12.52% and 15.35% did not disintegrate within 30 mins at pH 6.5. Therefore, lower coating percentages were tested. Sample at 7.91% coating showed the same trend, as burst time occurred between 50-60 mins at pH 6.5. Sample of 5.02% enteric coat showed a burst time of 60 mins at pH 6.0 and 35 mins at pH 6.5. Sample at 5.02% showed the most promising result, however burst time at pH 6.5 is still long and needs to be optimized.

3.6. Other Trials

COEP-055

The seal coating of formulation COEP-053 was kept for the manufacture of COEP-055. In lot COEP-055, the ratio of L30D55-FS30D used was 20:80 and the solid content was reduced to 10%.

TABLE 33
Enteric coating formulation of COEP-055

| Ingredients for enteric coat (COEP-055) | % w/w |
|---|---|
| Eudragit FS30D | 64.91 |
| Eudragit L30D55 | 16.23 |
| Plasacryl T20 | 6.69 |
| Talc | 12.17 |
| Total | 100 |

10% dispersion was prepared

TABLE 34
Coating parameters of COEP-055 seal coating and enteric coating

| Parameters | Seal (COEP-055) | Enteric (COEP-055) |
|---|---|---|
| Batch size (g) | 900 | 900 |
| Pan size (inches) | 15 | 15 |
| Pan speed (rpm) | 11.0-11.2 | 11.0-11.2 |
| Inlet temp (Actual ° C.) | 53.5-60.0 | 32.9-35.6 |
| Exhaust temp (° C.) | 40.7-42.5 | 26.3-31.8 |
| Spray rate (g/min) | 5.6-13.3 | 4.7-14.3 |
| Inlet air volume (cfm) | 199-202 | 200-207 |
| Atomizing air pressure (psi) | 20.0-20.3 | 19.4-19.9 |
| Air pattern pressure (psi) | 24.8-25.1 | 21.6-22.0 |
| Weight gain | 9.94% | 17.3% |

The 12.83% enteric coated sample was subjected to both a two-stage and a three-stage resistance study. The capsules were subjected to acid for 1 hour, followed by pH 6.5 burst time evaluation in the two-stage test. Burst time in pH 6.5 was observed to be greater than 45 mins. In the three-stage test, after 1 hour in acid the capsules were exposed to pH 6.0 for an hour and then subjected to pH 6.5. Burst in pH 6.5 occurred in less than 5 mins.

COEP-056

In lot COEP-056, the ratio of L30D55-FS30D used was 30:70.

The seal coating formulation was the same as COEP-055, but it was further diluted to 8% solid content and the weight gain achieved was lowered to 5%.

TABLE 35

| Ingredients for enteric coat (COEP-056) | % w/w |
|---|---|
| Eudragit FS30D | 55.96 |
| Eudragit L30D55 | 25.18 |
| Plasacryl T20 | 6.69 |
| Talc | 12.17 |
| Total | 100 |

A 10% dispersion was prepared

TABLE 36
Coating parameters of COEP-056 seal coating and enteric coating

| Parameters | Seal (COEP-056) | Enteric (COEP-056) |
|---|---|---|
| Batch size (g) | 900 | 850 |
| Pan size (inches) | 15 | 15 |
| Pan speed (rpm) | 11.1-13.2 | 12.2-13.2 |
| Inlet temp (Actual ° C.) | 52.7-55.4 | 32.1-34.2 |
| Exhaust temp (° C.) | 41.3-44.1 | 25.4-30.6 |
| Spray rate (g/min) | 9.8 | 10.0-15.0 |
| Inlet air volume (cfm) | 198-204 | 199-207 |
| Atomizing air pressure (psi) | 19.4-19.9 | 19.9-20.0 |
| Air pattern pressure (psi) | 24.5-24.9 | 21.9-22.2 |
| Weight gain | 5.38% | 19.56% |

The two-stage resistance test (1 hour in acid followed by burst time evaluation in pH 6.5) of capsules coated to 8.66% and 10.34% coating showed disintegration of 57 and 88 mins respectively, which was too long.

COEP-057

The seal coat formulation of COEP-057 was the same as lot COEP-055 (return to 9% solid content). The targeted weight gain also changed back to 10%. The enteric coating formulation was the same as COEP-056, but the solid content was decreased to 7.5% to get a more homogeneous coating and reduce variability.

TABLE 37
Coating parameters of COEP-057 seal coating and enteric coating

| Parameters | Seal (COEP-057) | Enteric (COEP-057) |
|---|---|---|
| Batch size (g) | 875 (“0”) + 25(“00”) | 836 + 14 |
| Pan size (inches) | 15 | 15 |
| Pan speed (rpm) | 11.0-12.2 | 11.1-16.3 |
| Inlet temp (Actual ° C.) | 50.3-52.1 | 32.0-33.9 |
| Exhaust temp (° C.) | 41.0-43.8 | 26.6-30.2 |
| Spray rate (g/min) | 4.9-9.8 | 9.95 |
| Inlet air volume (cfm) | 201-210 | 202-210 |
| Atomizing air pressure | 19.9-20.3 | 20.1-20.4 |
| Air pattern pressure (psi) | 24.6-24.9 | 21.9-22.0 |
| Weight gain | 10.1% | 9.41% (“00”) |

A few hand-filled size 00 capsules were incorporated into the pan with the size 0 capsules to evaluate their burst time. Samples coated to ˜7.5% were tested for burst time using a two-stage test (1 hour in acid followed by exposure to pH 6.0, 6.5 or 6.8). All capsules showed resistance in acid; however, burst time in pH 6.0, 6.5 or 6.8 was variable. Decrease in solid content and increase in pan speed did not improve the variability.

COEP-058

In order to decrease this variability and to obtain the lower desired burst time, the next coating trial was carried out only using one enteric polymer, L30D55. Proportions of Plasacryl and talc were also changed.

Again, the seal coat formulation was locked in and the same as COEP-055 and COEP-057 was used.

TABLE 38
Enteric coating formulation of COEP-058

| Ingredients for enteric coat (COEP-058) | % w/w |
|---|---|
| Eudragit L30D55 | 80.00 |
| Plasacryl T20 | 12.00 |
| Talc | 8.00 |
| Total | 100 |

A 15% dispersion was prepared

TABLE 39
Coating parameters of COEP-058 seal coating and enteric coating

| Parameters | Seal (COEP-058) | Enteric (COEP-058) |
|---|---|---|
| Batch size (g) | 850 | 850 |
| Pan size (inches) | 15 | 15 |
| Pan speed (rpm) | 11.1-16.4 | 11.1-16.3 |
| Inlet temp (Actual ° C.) | 51.8-55.0 | 27.0-33.0 |
| Exhaust temp (° C.) | 40.1-41.7 | 26.3-29.6 |
| Spray rate (g/min) | 7.9-13.7 | 7.9-9.9 |
| Inlet air volume (cfm) | 203-206 | 203-209 |
| Atomizing air pressure (psi) | 19.9-20.2 | 19.6-20.3 |
| Air pattern pressure (psi) | 24.9-25.1 | 21.7-22.1 |
| Weight gain | 10.0% | 16.85% |

The variability in the disintegration times between samples decreased (samples with 8.7616.85% coating tested), however, the coating % needed to be adjusted to achieve the target disintegration time. Between 8.76 and 16.85%, the disintegration burst time are between 20 and 35 mins at pH 6.8.

Optimizing Disintegration Burst Time (COEP-059, COEP-060, COEP-061)

Three lots were manufactured (COEP-059, COEP-060 and COEP-061) with exactly the same seal coat formulation and process parameters. Their enteric coating formulations and process parameters were also identical. The difference between these three was in their coating level, to achieve targeted disintegration time.

TABLE 40
Seal coating formulations of COEP-
059, COEP-060 and COEP-061

| | COEP-059 | COEP-060 | COEP-061 |
|---|---|---|---|
| Ingredients for seal coat | % w/w | % w/w | % w/w |
| HPMC E5 LV | 71.43 | 71.43 | 71.43 |
| Triethyl citrate | 7.14 | 7.14 | 7.14 |
| Talc | 21.43 | 21.43 | 21.43 |
| Total | 100 | 100 | 100 |

9% solid dispersion was prepared

For all three formulations, Plasacryl was increased from 15% (COEP-058) to 25% of the enteric polymer.

TABLE 41
Enteric coating formulations of
COEP-059, COEP-060 and COEP-061

| | COEP-059 | COEP-060 | COEP-061 |
|---|---|---|---|
| Ingredients for enteric coat | % w/w | % w/w | % w/w |
| Eudragit L30D55 | 74.08 | 74.08 | 74.08 |
| Plasacryl T20 | 18.52 | 18.52 | 18.52 |
| Talc | 7.40 | 7.40 | 7.40 |
| Total | 100 | 100 | 100 |

10% dispersions were prepared.

TABLE 42
Coating parameters of COEP-059, COEP-060 and
COEP-061 seal coatings and enteric coatings

| | Seal coatings | Enteric coatings | | |
|---|---|---|---|---|
| | Seal | Enteric | Enteric | Enteric |
| Parameters | (COEP-059, -060, -061) | COEP-059 | COEP-060 | COEP-061 |
| Batch size (g) | 850 | 850 | 850 | 837 (“0”) + 14 (“00”) |
| Pan size (inches) | 15 | 15 | 15 | 15 |
| Pan speed (rpm) | ≈13.0 | 13 | 13 | 13 |
| Inlet temp (Actual ° C.) | ≈55.0 | 32-34 | 30-33 | 32-33 |
| Exhaust temp (° C.) | ≈42.0 | 26-31 | 26-30 | 27-29 |
| Spray rate (g/min) | ≈9.7-16.6 | 7.8-13.7 | 9.4 | 9.4 |
| Inlet air volume (cfm) | ≈200 | ≈200 | ≈200 | ≈200 |
| Atomizing air | ≈20.0 | ≈19.5 | ≈19.5 | ≈20.0 |
| pressure (psi) | | | | |
| Air pattern | ≈24.0 | ≈22.0 | ≈22.0 | ≈22.0 |
| pressure (psi) | | | | |
| Weight gain | ≈10.0% | 22.68% | 11.97% | 10.48% |

For lot COEP-059, capsules with 10.78% coating were subjected to a two-stage resistance test; 1 hour in acid followed by exposure to pH 6.0, 6.5 or 6.8. All tested capsules were resistant for 60 mins in acid. The capsules had burst times of E35 mins in pH 6.0. Samples that were transferred to pH 6.5 and 6.8 had burst time of 31.8 (average n=2) and 29.3 mins (average n=2) respectively. The variability was minimal, but the enteric coating level needed to be adjusted slightly to achieve target disintegration time.

For lot COEP-060, capsules at 8.45% and 9.79% coating were evaluated. Tested samples resisted for 1 hour in acid, then both coating levels had a burst time of approximately 25-27 mins at pH 6.5. However, at pH 6.0 the 8.45% coated capsules had a burst time of 34 mins (average, n=3), when the 9.79% coated capsules had burst time of 73 mins (average, n=3). Therefore, 9.79% coating level was determined to be the best coating level to achieve target profile and more samples were tested with that coating level. Another 6 capsules of 9.79% coating sample tested in two-stage resistance test showed again burst time of 73 mins at pH 6.0, burst time of 31 mins in pH 6.5 and 28.7 mins (n=6) in pH 6.8. Variability of the results obtained was minimal and the results obtained with COEP-060 9.79% coating were close to targeted profile. The results were on the higher side of the disintegration time target, therefore a slight decrease in coating weight gain was attempted with lot COEP-061.

Samples of lot COEP-061 was taken to a weight gain of 9.35% (slightly lower than that in COEP-060) and disintegration time was evaluated using the same two-stage resistance method. Size “0” capsules and size “00” capsules were tested, and results are presented in the table below:

TABLE 43
disintegration time results obtained for lot COEP-061

| | 0.1N HCI | Burst time | | Burst time |
|---|---|---|---|---|
| | (pH 1.2) | pH 6.0 | | pH 6.5 |
| | for 60 mins | (n = 3) | | (n = 3) |
| COEP-061 | Resist (n = 3) | 58 ± 10 | mins | 29 ± 4 mins |
| (9.35% coated) - | | | | |
| Size “0” | | | | |
| COEP-061 | Resist (n = 3) | 65 ± 7 | mins | 27 ± 3 mins |
| (9.36% coated) - | | | | |
| Size “00” | | | | |

9.3% enteric coating was selected as the target coating level to achieve target disintegration profile.

4.4.7. Confirmation Lot Before Scale-Up (COEP-062)

Lot COEP-062 was manufactured using the same seal coating formulation, parameters and coating level as COEP-061. Few size “00” capsules were also added in the pan, to evaluate their burst time as well as for size “0” capsules.

All the capsules (size “00”) tested at 9.31% coating resisted 1 hour in acid. After, the samples were put in either pH 6.0, 6.5 or 6.8 buffers. Results obtained showed a burst time of 56±6 mins, 30±2 mins and 28±1 mins for pH 6.0, 6.5 and 6.8 respectively. Variability was minimal for results obtained and the results were similar to those of COEP-061. 9.3% coating was therefore targeted for scale-up trial.

Conclusion:
Coating formulations and process were successfully developed to coat capsules.
Capsules achieved acid resistance for at least 1 hour (pH 1.2) and exhibited rapid disintegration when the media was changed to pH 6.5 or pH 6.8 (≈≤30 mins).
Disintegration results obtained showed discriminatory bursts time between pH 6.0 and pH 6.5 (or 6.8).
Formulation of COEP-061 (and COEP-062) at 10% seal coat (Table 41) and 9.3% enteric coating (see Table 42) was selected to pursue scale-up trials.

### Example 4—Stability Tests

Preliminary stability testing showed that the capsules are stable for at least 12 months at 25° C. and at least 6 months under accelerated conditions (40° C.). All stability results were compliant with the specs.

Description

The appearance of ECC capsules will be evaluated at release and on stability as a general test of the product quality. The purpose of this test is to ensure that the drug product matches appearance and physical characteristics (shape and color) as described in the drug product specification.

Identification

Identification testing for cholestyramine will be conducted for the release of ECC capsules to establish the identity of the drug substance in the drug product in a specific way. Testing is performed by an in-house ATR infrared (IR) spectroscopy method Identification is positive if the IR spectrum of the sample corresponds to that of the cholestyramine resin reference standard. Pre-validation of this method for specificity has shown that the method is suitable for identification of cholestyramine resin in ECC capsules.

Assay

As specified in the ICH guideline Q6A, a specific, stability-indicating procedure is included to determine the content (strength) of cholestyramine in ECC capsules at release and during stability testing. The in-house UPLC method has been pre-validated to demonstrate system suitability, specificity, linearity, accuracy, precision (repeatability, intermediate precision) and stability of sample and standard solutions. Limits of 85.0-115.0% of the label claim have been set based on the USP and BP monographs for powder dosage form. Results from the Phase I clinical batch have met this specification, which further supports this criterion.

Uniformity of Dosage Units

Uniformity of dosage units testing will be conducted for the release of ECC capsules to confirm the uniformity of the drug substance throughout the batch. This test complies with the USP chapter <905> for the uniformity of dosage units. Since the amount of drug substance in the drug product is more than 25 mg and it represents more than 25% of the capsule weight, testing is performed by Weight variation.

Disintegration

Disintegration testing to evaluate capsule burst time is conducted for release and stability of ECC capsules to report the delayed release characteristics of the drug product performance. Testing is performed using a Pharmascience in-house method. This test measures the time it takes for the capsules to burst after immersion in a disintegration bath of acid media (0.1N), for up to 1 hour, followed by immersion in potassium phosphate buffer (pH 6.8) for up to 60 minutes. Capsule burst time will be evaluated by monitoring each vessel for signs of capsule and resin release. The capsule burst time is expected to be about 20 minutes. ECC capsules are seal-coated and then enterically coated with pH-dependent polymers that have a pH solubility trigger of 5.5. During development work, the amount of coating was adjusted to achieve target capsule burst times of NLT 60 minutes at pH 1.2, NLT 45 minutes at pH 6.0, and NMT 30 minutes at pH 6.8, using USP Apparatus 3. This aligns with the targeted clinical efficacy and safety profile of cholestyramine release in the approximate pH range of 6.5-6.8 as mirrored in the environment of the mid-jejunum to ileum. This allows cholestyramine release to bypass the duodenum (thus preventing or reducing the magnitude of potential drug-drug interactions) while enabling binding of bile acids prior to the colon (thus preventing or reducing bild-acid induced diarrhea). At this stage of clinical development, testing is performed for characterization only. Validation is not applicable to this method. The disintegration test limits for release and stability purposes will be established pending generation of more data from subsequent clinical batches.

Styrene

This test is included in the specification to control the presence of the impurity Styrene in the drug product at release and under stability. Testing is performed using a Pharmascience in-house HPLC method which has been pre-validated for system suitability, specificity, linearity, accuracy, precision (repeatability), and stability of standard solutions. The limit is NMT 1 ppm as per the BP monograph for Colestyramine Oral Powder. Results from the Phase I clinical batch have met this specification, which further supports this criterion.

Microbial Limit Tests

These tests are performed at release and under stability to monitor the microbiological quality of the drug product. The limits for total aerobic microbial count and total yeasts and molds count (evaluated using USP chapter <61>) and for Escherichia coli (evaluated using USP chapter <62>) are set in accordance with USP chapter <1111> for the microbiological examination of nonsterile products.

### Example 5—Scale-Up Manufacture

Lots: COEP-069, COEP-070, COEP-071 & COEP-072 (700764, 700765, 700766 & 700816)

Once the cholestyramine 425 mg EC capsules have been manufactured on a lab scale, scale-up manufacture was achieved as per the following process and analytical results.

Pilot bio lots (lot P-2637 and P-2639) were manufactured at 2.0 kg scale and tested for both scintigraphy and drug-drug interaction (DDI) studies.

According to the results it was decided to select the similar formulation composition as lot P-2639 (With 6.5% EC) for Phase-II trials. The only difference in formulation between P-2639 and the present scale-up (lot 700766) is the removal of samarium oxide from formula P-2639 (replaced by Lactose). Lot 700764/COEP-069 (cholestyramine blend) was prepared at a 120 kg scale, and then it was encapsulated (700765/COEP-070) in HPMC size “00” capsules (on large scale equipment—Bosch GKF400). 20 kg of capsules were used for each pan load to establish scale up coating parameters and 3 coating trials were performed (COEP-071 fraction #1 & #2 and COEP-072).

The process of the EC capsules comprises the following major steps:
Blending
Encapsulation
Sealing
Enteric coating

1. Blending of the Formulation

1.1. Detailed Flow Process Chart

1.2 Formulation Composition

TABLE 44
Blending (LOT NO: 700764/COEP069):

| | | | | | Theoretical | |
|---|---|---|---|---|---|---|
| Item | Qty/cap | | DESCRIPTION | % w/w | Quantity | |
| 1 | 425.0 | mg | CHOLESTYRAMINE | 85.00 | 102000 | g |
| | | | RESIN USP | | | |
| 2 | 70.1 | mg | LACTOSE | 14.02 | 16824 | g |
| | | | MONOHYDRATE | | | |
| | | | SUPERTAB11SD EP | | | |
| 3 | 2.4 | mg | COLLOIDAL | 0.48 | 576 | g |
| | | | SILICON | | | |
| | | | DIOXIDE | | | |
| 4 | 2.5 | mg | MAGNESIUM | 0.50 | 600 | g |
| | | | STEARATE NF/BP | | | |
| S- | 500.0 | mg | | 100.00 | 120000 | g |
| TOTAL | | | | | | |

1.3 Comments on the Formulation Composition

The process consists of mixing together in the 500L bin Cholestyramine, Lactose, Colloidal silicon followed by milling, re-mixing and lubrication with Mg stearate. The final mix Bin filling level is approx. 60%. The yield is 100%.

2. Encapsulation

2.1 Encapsulation Process

120 kg of cholestyramine blend from lot COEP-069 were used for encapsulation in Bosch GKF400 with size 00 capsule change parts.

TABLE 45

| | 700764/ |
|---|---|
| Lot # | COEP-070 |
| Equipment | Bosch |
| | GKF400 |
| Number of stations | 12 (n = 36) |
| Filling speed (cycles/min) | 72 |
| Detector filling height (mm) | 25 |
| Height Bloc 1 (mm) | 12 |
| Height Bloc 2 (mm) | 10 |
| Height Bloc 3 (mm) | 12 |
| Height Bloc 4 (mm) | 10 |
| Height Bloc 5 (mm) | 5 |
| Dosing disk | #00 |
| Dosing disk height (mm) | 21.3 |
| Capsule size | “00” |

2.2 Results

TABLE 46
Encapsulation Lot: 700765/COEP-070:

| | | | | Theoretical | |
|---|---|---|---|---|---|
| Item | Qty/cap | DESCRIPTION | % w/w | Qty | |
| 1 | 500.0 | R&D IN | −80.27% | 120000 | g |
| | | CHOLESTYRAMINE | | | |
| | | CAPS BLEND | | | |
| 2 | 122.9 | HPMC EMPTY | −18.73% | 29496 | g |
| | | CAPSULE SHELL | | | |
| | | SIZE#00 | | | |
| S- | 622.9 mg | | | 149496 | g |
| TOTAL | | | | | |

TABLE 47

| Lot # | COEP-070 | |
|---|---|---|
| Empty capsule weight (mg) | 122.83 | |
| Fill weight (mg) | 500 | mg |
| Total capsule weight (mg) | 622.83 | mg |

2.3 Comments and Observations on Encapsulation
Excellent flow. Target fill weight of 500 mg per capsule was achieved.
Empty capsules weight variation is approximately 0.66%
Individual filled weight variation is approximately 0.71
Visual inspection allows to ensure proper closure quality.

3. Sealing Coating

3.1 Process

Cholestyramine capsules from lot COEP-070 were used for seal coating. A 30 inch pan was used where a load of 20 kg could be accommodated. Three 20 kg fractions were coated.

TABLE 48

| | 700766/ | | 700766/ | |
|---|---|---|---|---|
| | COEP-071 | | COEP-071 | 700816/ |
| Lot # | (Fraction-1) | | (Fraction-2) | COEP-072 |
| Capsule size | Size “00” | | Size “00” | Size “00” |
| Coating parameters | | | | |
| Pan size | 30″ | | 30″ | 30″ |
| Batch size | 20 | kg | 20 kg | 20 kg |
| No of guns | 2 | | 2 | 2 |
| Gun to bed distance | 13 cm ± 2 cm | | 13 cm ± 2 cm | 13 cm ± 2 cm |
| Pan speed (rpm) | 12-14 | | 12-13 | 13-16 |
| Atomizing air pressure | 40 | | 40 | 40 |
| Pattern air pressure | 42-45 | | 42 | 42 |
| Inlet air temperature | 58-65 | | 58-65 | 60-98 |
| (° C.) | | | | |
| Exhaust temperature | 43-44 | | 42-44 | 38-45 |
| Air volume (cfm) | ≈480 | | ≈480 | 400-456 |
| Spray rate (g/min) | 65-110 | | 90-110 | 96-129 |
| % weight gain | 10.0 | | 9.9 | 10.1 |
| Drying time | n/a | | n/a | n/a |
| Coating time (Hrs) | 4 | hrs | 3 hr 30 min | 2 hr 45 min |

| | | | |
|---|---|---|---|
| Solid content (%) | 9% | 9% | 9% |

3.2 Results

### Coating Lot No: 700766:

Seal Coating: same as in Table 28

### Pan Loading: 20 kg

Fractions: Two (2 Pan loads)
Coating to achieve 10% weight gain. A 9% w/w suspension is prepared.

TABLE 49

| | | | | | Theoretical | |
|---|---|---|---|---|---|---|
| Item | Qty/cap | | DESCRIPTION | % w/w | Qty** | |
| 1 | 622.9 | mg | R&D BU CHOLESTYRAMINE 425 | 90.91 | 15000 | g |
| | | | MG CAPS | | | |
| 2 | 44.5 | mg | HYPROMELLOSE 2910 | 6.49 (71.41)*** | 1071.4 | g |
| 3 | 4.45 | mg | TRIETHYL CITRATE (7.14% of seal | 0.65 (7.14)*** | 107.1 | g |
| | | | coat only) | | | |
| 4 | 13.35 | mg | TALC USP/BP LO-MICRO | 1.95 (21.43)*** | 321.4 | g |
| 5 | — | | Purified Water, USP (EDP) 85013* | — | 8986.4 | g |
| 6 | — | | Purified Water, USP (EDP) 85013* | — | 3483.7 | g |
| 7 | — | | Purified Water, USP (EDP) 85013* | | 2696.6 | g |
| | | | | | 1500 | g solids |
| Total | 685.2 | mg | | 100.00 | 16667 | g total |

*Not present in final formulation
**Theoretical quantity is based on a 15 kg pan load. Pan load was then adjusted to 20 kg for proper fill, but initial excess of 50% coating dispersion was enough to accommodate 20 kg batch.
***% based on seal coat content only

3.3. Comments

In order to maintain a constant outlet of 43-44° C. while spraying, Inlet temperature is set at 58−65° C. depending on the spray rate used.
COEP-072 was planned to challenge the spray rate to obtain a better and more uniform film that would help unite the body with the cap and improve acid resistance. For the seal coating of fraction 3 the spray rate was increased up to 173 g/min without observing sticking. However, this rate was not maintained because the Labcoat was not able to maintain a product temperature of 43±1° C. the spray rate was then reduced to 129 g/min and the temperature at 43±1° C. could be maintained. At the end of coating of COEP-072, no improvement was seen. The body and cap were not stuck.

4. Enteric Coating

4.1 Enteric coating process

A 10% w/w suspension is prepared.
Pan load: ^{˜}22 kg

TABLE 50

| | COEP-071 | COEP-071 | | |
|---|---|---|---|---|
| Lot# | (Fraction-1) | (Fraction-2) | COEP-072 | |
| Capsule size | Size “00” | Size “00” | Size “00” | |
| Coating parameters | | | | |
| Pan size | 30″ | 30″ | 30″ | |
| Batch size | 22 kg | 22 kg | 22 | kg |
| No of guns | 2 | 2 | 2 | |
| Gun to bed distance | 13 cm ± 2 cm | 13 cm ± 2 cm | 13 cm ± 2 cm | |
| Pan speed (rpm) | 13-15 | 14-15 | 13-15 | |
| Atomizing air pressure | 45 | 45 | 40-45 | |
| Pattern air pressure | 35 | 35 | 35 | |
| Inlet air temperature | 40-49 | 47-50 | 50-64 | |
| (° C.) | | | | |
| Exhaust temperature | 29-30 | 28-30 | 29-32.5 | |
| Air volume (cfm) | ≈450 | ≈470 | ≈450 | |
| Spray rate (g/min) | 58-100 | 89-101 | 100-200 | |
| % weight gain | 10.5 | 6.5 | 6.5 | |
| Drying time | n/a | n/a | n/a | |
| Coating time (Hrs) | 4 hrs 15 min | 2 hr 30 min | 47 | min |

| | | | |
|---|---|---|---|
| Solid content (%) | 10% | 10% | 10% |

4.2. Enteric Coating Results

### Fractions: Two (2 Pan Loads)—Fraction 1 Coated to 10.5% WG/Fraction 2 Coated to 6.5% WG

TABLE 51

| | Qty / | | | | Theoretical | |
|---|---|---|---|---|---|---|
| Item | cap | | DESCRIPTION | % w/w | Qty** | |
| 8 | 685.2 | mg | CHOLESTYRAMINE 425MG CAPS | 93.91 | 16500 | |
| | | | (seal coated) | | | |
| 9 | 33.0 | mg | METHACRYLIC ACID COPOLYMER | 4.52 | 2648.2 | g |
| | | | DISPERSION (Poly(methacrylic acid-co- | | | |
| | | | ethyl acrylate) 1:1; L30D55) | | | |
| | | | (30% solid dispersion) | | | |
| 10 | 8.2 | mg | PLASACRYL T20 | 1.12 | 993.1 | g |
| | | | (20% solid dispersion. Glyceryl | | | |
| | | | monostearate (GMS), TEC and | | | |
| | | | polysorbate 80) | | | |
| 11 | 3.3 | mg | TALC USP/BP LO-MICRON | 0.45 | 79.4 | g |
| 12 | — | | Purified Water, USP (EDP) 85013* | | 3907.2 | g |
| 13 | — | | Purified Water, USP (EDP) 85013* | | 1858.3 | g |
| 14 | — | | Purified Water, USP (EDP) 85013* | | 1238.9 | g |
| Total | 729.7 | mg | | 100.00 | 1072.5 | g solids |

*Not present in final formulation
**Enteric coating dispersion was prepared based on 16.5 kg pan load, but initial excess of 50% coating dispersion was enough to accommodate 22 kg batch.

During enteric coating, capsules at different enteric coating levels were collected to determine the optimal weight gain based on disintegration time results: Samples were collected at: 5.5%, 6.4%, 6.5%, 7.5%, 9.0%, 10.5%

4.3 Comments on the Enteric Coating
To maintain a constant outlet of 30±1° C. while spraying, Inlet temperature is set at 40-48° C. depending on the spray rate used.
Curing is done during 30 mins at 30° C.

5. Results

5.1 Analytical Method (Disintegration)

Since, there are no reference products or efficacy data on the proposed drug product, preliminary target disintegration times were established based on scientific reasoning. To evaluate the performance of the drug product in vitro, a disintegration method is used in different pH media.
Two methods were used to evaluate the cholestyramine enteric coated capsules.

TABLE 52

| | | Method-2 |
|---|---|---|
| | Method-1 | (Release method) |
| | (Apparatus-3), 10 | (DT Apparatus), 30 |
| | dps/min | dps/min |
| Acid Phase | 0.1N HCL, 1 hr | 0.1N HCL, 1 hr |
| Buffer Phase | Phosphate buffer pH 6.0 | Phosphate buffer pH 6.8 |

5.2 Disintegration Burst Time Results

I. Disintegration Testing with USP Apparatus-3:

TABLE 53

| | COEP-071 fraction 1; 6.4% EC | | COEP-071 fraction 2; 6.5% EC | |
|---|---|---|---|---|
| | Method: Apparatus-3 | | | |
| | | Burst time (min) | | Burst time (min) |
| Capsules | Acid | (pH 6.0) | Acid | (pH 6.0) |
| 1 | conforms | Burst @ approx. | conforms | Burst @ approx. |
| | | 47.0 min | | 55 min |
| 2 | conforms | Burst @ approx. | conforms | Burst @ approx. |
| | | 18 min | | 57 min |
| 3 | conforms | Burst @ approx. | conforms | Burst @ approx. |
| | | 50 min | | 54 min |
| 4 | conforms | >45 min; (Burst not | conforms | >45 min; (Burst |
| | | observed/Photos | | not observed) |
| | | taken) | | |
| 5 | conforms | >45 min; (Burst not | conforms | >45 min; (Burst |
| | | observed/Photos | | not observed) |
| | | taken) | | |
| 6 | conforms | >45 min; (Burst not | conforms | >45 min; (Burst |
| | | observed/Photos | | not observed) |
| | | taken) | | |

Note that lot P-2639 had acid resistance and a burst time of 40.0±6.8 mins at pH 6.0.
II. Disintegration Testing with DT Apparatus:

TABLE 54

| | COEP-071 fraction 1; 6.4% EC | | COEP-071 fraction 2; 6.5% EC | |
|---|---|---|---|---|
| | Method: DT Apparatus | | | |
| | | Burst time (min) | | Burst time (min) |
| Capsules | Acid | (pH 6.8) | Acid | (pH 6.8) |
| 1 | conforms | <20 min; (Burst @ | conforms | <20 min; (Burst |
| | | approx. 14.0 min) | | @ approx. 15.0 min) |
| 2 | conforms | <20 min; (Burst @ | conforms | <20 min; (Burst |
| | | approx. 15.5 min) | | @ approx. 15.5 min) |
| 3 | conforms | <20 min; (Burst @ | conforms | <20 min; (Burst |
| | | approx. 14.0 min) | | @ approx. 16.0 min) |
| 4 | conforms | <20 min; (Burst @ | conforms | <20 min; (Burst |
| | | approx. 13.5 min) | | @ approx. 9.5 min) |
| 5 | conforms | <20 min; (Burst @ | conforms | <20 min; (Burst |
| | | approx. 7.5 min) | | @ approx. 9.5 min) |
| 6 | conforms | <20 min; (Burst | conforms | <20 min; (Burst |
| | | @ approx. 8.0 min) | | @ approx. 12.0 min) |

Note that lot P-2639 had acid resistance and a burst time of 24.0 ± 2.2 mins at pH 6.8

TABLE 55

COEP-071 fraction 1; 10.5% EC
Method: DT Apparatus

| Capsules | Acid | Burst time (min) (pH 6.8) |
|---|---|---|
| 1 | conforms | <20 min; (Burst @ approx. |
| | | 18.0 min) |
| 2 | conforms | <20 min; (Burst @ approx. |
| | | 18 min) |

Note:
Lot 700816/COEP-072 capsules were not tested due to the deformation & over wetting of capsules observed during enteric coating

6.5% EC weight gain capsules meet the criteria of disintegration (Fraction 1 & Fraction 2)
Based on the scale up batch, the spray rate of 80 g/min (70-90 g/min) for POC batch was used.

6. Placebo Selection for Phase II Studies:
Microcrystalline cellulose and compressible sugar were proposed to replace cholestyramine in the placebo formulation. Thus, these excipients were tested for bile acid binding properties (Assay Testing—Binding of GCA).
Since, MCC possesses similar material characteristics (bulk density, fill volume etc.) as that of cholestyramine, Microcrystalline cellulose was selected to replace API in the placebo formulation
Microcrystalline cellulose is an insoluble excipient, just like cholestyramine is insoluble, which makes it a very similar replacement for placebo.
Microcrystalline cellulose PH 102 grade was selected.

TABLE 56
Summary of the formulations herein developed and tested in vivo

| | | |
|---|---|---|
| Scintigraphy | DDI | Phase II trial (Scale up |

Capsule Core

| | | | | | | |
|---|---|---|---|---|---|---|
| Cholestyramine Resin | 85.00% | (425 mg) | 85.00% | (425 mg) | 85.00% | (425 mg) |
| (active ingredient) | | | | | | |
| Lactose monohydrate | 13.42% | (67.1 mg) | 14.02% | (70.1 mg) | 14.02% | (70.1 mg) |
| (diluent) | | | | | | |
| Colloidal silicon | 0.48% | (2.4 mg) | 0.48% | (2.4 mg) | 0.48% | (2.4 mg) |
| dioxide (glidant) | | | | | | |
| Magnesium stearate | 0.5% | (2.5 mg) | 0.5% | (2.5 mg) | 0.5% | (2.5 mg) |
| (lubricant) | | | | | | |

| | | | |
|---|---|---|---|
| Samarium oxide | 0.6% | — | — |

Sealing coat

| | | | | | | |
|---|---|---|---|---|---|---|
| HPMC (Hypromellose) | 71.43% | (44.5 mg) | 71.43% | (44.5 mg) | 71.43% | (44.5 mg) |
| (film former) | | | | | | |
| Triethyl citrate (TEC) | 7.14% | (4.4 mg) | 7.14% | (4.4 mg) | 7.14% | (4.4 mg) |
| (plasticizer) | | | | | | |
| Talc (Lo-micron) | 21.43% | (13.3 mg) | 21.43% | (13.3 mg) | 21.43% | (13.3 mg) |
| (anti-adherent, filler of | | | | | | |
| gaps) | | | | | | |

| | | | |
|---|---|---|---|
| Seal coating weight | 10% | 10% | 10% |
| gain | | | |

Enteric coating

| | | | | | |
|---|---|---|---|---|---|
| Methacrylic acid | 74.08 | (30.5 mg) | 74.08 | (30.5 mg) | 74.16% |
| copolymer (Eudragit ™ | | | | | |
| L30D-55, 30% solid | | | | | |
| dispersion) | | | | | |
| Plasacryl ™ T20 (20% | 18.52% | (7.6 mg) | 18.52% | (7.6 mg) | 18.42% |
| solid dispersion) | | | | | |
| Talc (Lo-micron) | 7.41% | (3.0 mg) | 7.41% | (3.0 mg) | 7.42% |
| (anti-adherent) | | | | | |

| | | | |
|---|---|---|---|
| Enteric coating weight | 6.5% | 6.0% | — |
| gain (%) | | | |
| Enteric coating | 6.5% | 6.0% | 6.5% |
| weight gain | | | |

### Example 6—Scintigraphy Study in 8 Healthy Adult Male Subjects (Age Between 18-50 Years; Weight Between 65-100 kg; Height Between 160-200 cm)

The anatomical site of release of cholestyramine is of paramount importance for the proposed indication since the drug must be available for non-reabsorbed bile acids binding prior to their passage into the colon. At the same time, a too proximal delivery of the resin should be avoided, in order to prevent any potential drug interaction in the duodenum and to preserve the lipolytic activity of bile acids.

This Phase I study was an open-label, single center, single-dose, non-randomized study conducted to evaluate the gastrointestinal transit, site of disintegration, site of dispersion and associated variability of a coated HMPC cholestyramine capsule technology and to assess its safety and tolerability, before being used for the symptomatic control of bile acid diarrhea due to Short Bowel Syndrome (SBS). The study assessed the time and gastrointestinal location of capsule release at the Proximal Small Intestine (PSI), Mid Small Intestine (MSI), Distal Small Intestine (DSI) or Terminal Ileum (TI) where PSI approximates the duodenum to the mid jejunum; the MSI is approximately the distal jejunum to the proximal ileum; the DSI is approximately the mid ileum to distal ileum and the TI is the segment of the ileum just prior to the cecum/ascending colon.

The Coated HPMC Cholestyramine Capsule Technology consisted of an HPMC capsule coated with a polymer intended to release the capsule contents (cholestyramine) in the ileum portion of the small intestine at a pH>6.2.

Non-radioactive samarium oxide was incorporated in the powder blend used to fill the Coated HPMC Cholestyramine Capsule Technology at the time of manufacture. The non-radioactive isotope samarium-152 was subsequently converted to the radioactive isotope samarium-153 by a short exposure to a neutron flux. External gamma scintigraphy was used to monitor the gastrointestinal transit, site of disintegration and dispersion of the Coated HPMC Cholestyramine Capsule Technology. The water used to swallow the test formulation did not contain radioactivity. The capsules were to be swallowed whole and not be chewed or crushed.

The test formulation contained 425 mg cholestyramine as the active ingredient, formulated in size “00” capsules, said capsule being coated with an enteric polymer. On the day prior to the administration of Treatment A (Day −1 of Dosing Period 1) each subject was administered radioactive Tc-99m DTPA in 240 mL of water for the purpose of delineating gastrointestinal anatomy by gamma scintigraphy. On Day 1 of each Dosing Period each subject was administered the Coated HPMC Cholestyramine Capsule Technology and gamma scintigraphy was performed.

Gastrointestinal residence values post dose are listed in Table 57. The time and gastrointestinal location of capsule release and the subsequent exposure time in the small intestine after capsule rupture are listed in Table 58. Subject 007 was excluded from the mean analysis due to capsule release initiating while still in the stomach.

Of the seven evaluable subjects (omitting subject 007), the site of capsule release occurred two times in the proximal small intestine (001 and 006), three times in the mid small intestine region (002, 003 and 004) and two times in the distal small intestine (005 and 008).

For the two subjects 001 and 006 where the 1st release was observed in the proximal small intestine both exhibited prolonged residence time in this earliest segment of the small intestine which contributed to release of the cholestyramine resin occurring in the PSI. The longer lag phase in the small intestine of 1.55 hours observed in subject 006 for capsule rupture to initiate (Table 47) is also indicative of the lower pH in this segment of the small intestine (pH=″5.5-″6.5) as compared to more distal segments of the small intestine, and furthermore, this longer disintegration time correlates with the longer in vitro capsule disintegration time at pH 6 as compared to pH 6.8.

Subjects 002, 003 and 004 demonstrated capsule release in the mid small intestine which approximately represents the transition from the distal jejunum to the proximal ileum regions. For subjects 002 and 004, the study showed the transition of the released radioactive marker from the proximal small intestine to distal small intestine region following capsule rupture. The other mid small intestine release observed in subject 003 was characterized by the intact capsule arriving to the proximal ileal region with an interpolated time of release assigned as 1.65 hours and the dispersed material remaining in this region through approximately the 3.51 hours image.

Release to the distal small intestine was achieved in subjects 005 and 008. In each of these subjects, the movement of the intact capsule through the proximal and mid small intestine was rapid and the capsule reached the distal small intestine within 30-45 mins after gastric emptying. Since the pH in the distal ileum is typically higher (pH=7-7.5), capsule rupture may also be enhanced even though dispersion of the released material becomes less pronounced due to decreased motility in the distal and terminal ileum since this region of the small intestine acts an area of concentration prior to movement of foodstuff into the cecum/colon.

In conclusion of this study, the EC-cholestyramine Capsule, 425 mg, successfully targeted the small intestine and provided gastric resistance in 7 of 8 subjects. The single failure to acid resistance occurred in a subject showing delayed gastric residence (>3.5 hours) in the fasted condition. Improved acid resistance can likely be achieved with banding of the cap and body. The time of capsule release upon entering the small intestine was tightly clustered (range 0.75 to 1.55 hours, mean=1.05±0.26 hours). It is anticipated that this lag phase plus the additional delay from gastric emptying is an adequate time interval to avoid drug-drug interactions with immediate release drug products that are potentially co-administered with the EC-cholestyramine capsule.

TABLE 57
Gastrointestinal residence values post dose

| | Gastric Residence Values | | Area Under the SITT Curves | | | ATC50% and SITT50% (hrs) | |
|---|---|---|---|---|---|---|---|
| | GE | AUC GE | | | Total | ATC50% | SITT50% |
| Subject | (hrs) | Curve | PSI | DSI | SI | (hrs) | (hrs) |
| 001 | 0.23 | 0.160 | 2.382 | 1.593 | 3.975 | 3.99 | 3.76 |
| 002 | 0.37 | 0.352 | 1.153 | 1.615 | 2.768 | 3.12 | 2.75 |
| 003 | 0.47 | 0.370 | 0.151 | 4.765 | 4.916 | 4.48 | 4.01 |
| 004 | 0.48 | 0.504 | 2.322 | 3.388 | 5.710 | 6.10 | 5.62 |
| 005 | 2.63 | 2.615 | 0.053 | 1.536 | 1.589 | 4.13 | 1.50 |
| 006 | 0.82 | 0.791 | 2.676 | 0.919 | 3.595 | 4.39 | 3.57 |
| 008 | 0.38 | 0.368 | 0.254 | 3.308 | 3.562 | 3.97 | 3.59 |
| Mean | 0.77 | 0.737 | 1.284 | 2.446 | 3.731 | 4.31 | 3.54 |
| SD | 0.84 | 0.850 | 1.162 | 1.390 | 1.350 | 0.90 | 1.25 |
| RSD (%) | 109.3% | 115.3% | 90.4% | 56.8% | 36.2% | 21.0% | 35.3% |
| Median | 0.47 | 0.370 | 1.153 | 1.615 | 3.595 | 4.13 | 3.59 |
| Minimum | 0.23 | 0.160 | 0.053 | 0.919 | 1.589 | 3.12 | 1.50 |
| Maximum | 2.63 | 2.615 | 2.676 | 4.765 | 5.710 | 6.10 | 5.62 |
| 007 ⁽¹⁾ | 3.48 | 3.525 | 0.721 | 2.845 | 3.566 | 7.20 | 3.72 |

NOTES:
⁽¹⁾ Subject 007 was excluded from the summary statistics due to capsule release occurring in the stomach
Abbreviations: GE = gastric emptying; PSI = proximal small intestine; MSI = mid small intestine; DSI = distal small intestine; SI = small intestine; ATC = arrival time at the cecum/colon; SITT = small intestine transit time
PSI approximates the duodenum to the mid jejunum; MSI approximates the distal jejunum to proximal ileum; DSI approximates the mid ileum to distal ileum; the terminal ileum is the segment of the ileum immediately preceding the cecum/ascending colon
SITT50% = ATC50%-GE

TABLE 58
Time and gastrointestinal location of capsule release and exposure
time in the small intestine after release of capsule contents.

| | Capsule Release Time and | | | Exposure Time in Small Intestine Post | | | |
|---|---|---|---|---|---|---|---|
| | Gastrointestinal Location | | | Capsule Release | | | |
| | | Time in SI | GI | SITT50 | | | AUC |
| | 1^{5t} | until I^{e} | Location | Post | AUC PSI | AUC DSI | Total SI |
| | Release | Release | of I^{e} | Release | Post | Post | Post |
| Subject | (hr) | (hr) | Release | (hrs) | Release | Release | Release |
| 001 | 1.27 | 1.03 | PSI | 2.72 | 1.256 | 1.593 | 2.849 |
| 002 | 1.29 | 0.92 | MSI | 1.83 | 0.222 | 1.612 | 1.834 |
| 003 | 1.65 | 1.18 | MSI | 2.83 | 0.000 | 3.552 | 3.552 |
| 004 | 1.40 | 0.92 | MSI | 4.70 | 1.333 | 3.388 | 4.721 |
| 005 | 3.63 | 1.00 | DSI | 0.50 | 0.000 | 0.487 | 0.487 |
| 006 | 2.37 | 1.55 | PSI | 2.02 | 0.976 | 0.919 | 1.895 |
| 008 | 1.13 | 0.75 | DSI | 2.84 | 0.000 | 2.691 | 2.691 |
| Mean | 1.82 | 1.05 | | 2.49 | 0.541 | 2.035 | 2.576 |
| SD | 0.90 | 0.26 | | 1.28 | 0.620 | 1.196 | 1.355 |
| RSD (%) | 49.4% | 24.4% | | 51.3% | 114.6% | 58.8% | 52.6% |
| Median | 1.40 | 1.00 | | 2.72 | 0.222 | 1.61 | 2.691 |
| Minimum | 1.13 | 0.75 | | 0.50 | 0.000 | 0.49 | 0.487 |
| Maximum | 3.63 | 1.55 | | 4.70 | 1.333 | 3.55 | 4.721 |
| 007 ⁽¹⁾ | 3.10 | Not applicable | Stomach | 4.10 | 0.721 | 2.845 | 3.566 |

NOTES:
⁽¹⁾ Subject007 was excluded from the summary statistics due to capsule release occurring in the stomach
Abbreviations: GI = gastrointestinal; PSI = proximal small intestine; MSI = mid small intestine; DSI = distal small intestine; SI = small intestine; ATC = arrivai time at the cecum/colon; SITT = small intestine transit time
PSI approximates the duodenum to the mid jejunum; MSI approximates the distal jejunum to proximal ileum; DSI approximates the mid ileum to distal ileum; the terminal ileum is the segment of the ileum immediately preceding the cecum/ascending colon.
Time in SI until 1^{5t} Release = Time of 1^{5t} Capsule Release/Rupture-Gastric Emptying time of Capsule SITT50% Post Release = ATC50%-Time of 1^{5t} Capsule Release/Rupture

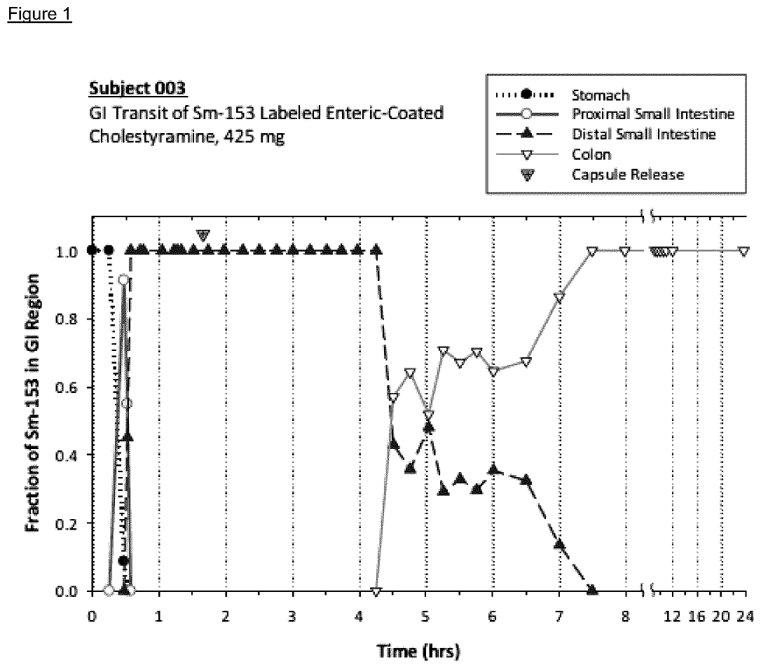

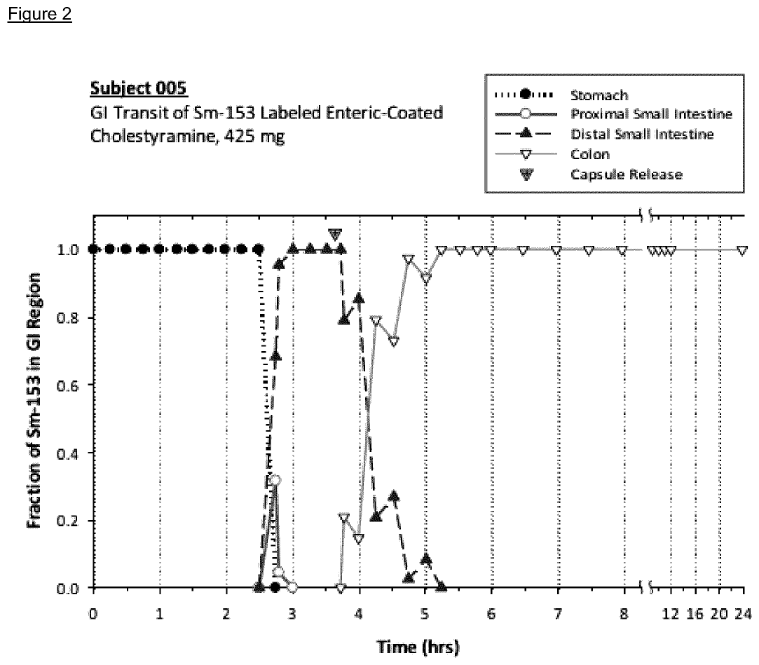

The gastrointestinal transit profiles and time of capsule releases following the oral administration of an enteric coated cholestyramine capsule (425 mg) radiolabeled with samarium-153, during the scintigraphy study of Subjects 003 and 005 are reported in FIGS. 1 and 2.

Safety data from 8 healthy volunteers participating in a single dose scintigraphic evaluation of ECC capsules showed that the product was well tolerated, and its safety profile was very good.

On the other hand, from a pharmacokinetic point of view, this study confirmed that the enteric-coating of the cholestyramine capsules resists disintegration under acidic pH conditions in vivo and enables successful targeting and delayed release of cholestyramine close to or in the ileum, i.e. in the targeted area. Consequently, this formulation is adequate for further clinical exploration.

### Example 7—Single Dose Crossover Comparative Bioavailability Study to Assess the Impact of a New Cholestyramine Enteric Coated Capsule on the Pharmacokinetics of HCTZ Compared to Cholestyramine Regular Powder in Healthy Male and Female Volunteers/Fasting State

Drug-drug interactions (DDI) are complex and have proven to be a major challenge for health-care providers. One of the questions that must be addressed before new drugs can be safely administered is whether there is a drug interaction with other medications taken by the patient for the treatment of co-morbidities. Therefore, the interaction potential for a new compound is regularly evaluated during the clinical drug development.

In order to document the partial or full prevention of drug-drug interactions with the use of the new enteric-coated cholestyramine capsules compared to commercially available cholestyramine powder suspension, a Phase I drug-drug interaction (DDI) study was conducted to assess whether the targeted delivery of cholestyramine to the mid- to distal small intestine reduces the risk of drug interaction with hydrochlorothiazide (HCTZ), used as a representative victim drug. HCTZ is a diuretic and an antihypertensive agent. HCTZ is rapidly and almost completely absorbed from the gastrointestinal tract, particular the proximal intestine. The onset of action after oral administration occurs in 2 hours and the peak effect at approximately 4 hours. The duration of action persists for approximately 6 to 12 hours. The study investigated the effect of ECC capsules on plasma kinetics of HCTZ in healthy volunteers. The objective was to demonstrate the absence of such a drug-drug interaction between ECC capsules and HCTZ, under fasting conditions.

Cholestyramine powder is known to substantially reduce bioavailability of HCTZ as measured by plasma levels and urinary excretion of HCTZ. The effects of this drug-drug interaction have been shown to be time-dependent and heightened by multiple dosing. In order to demonstrate the absence of such a drug-drug interaction between ECC capsules and HCTZ, due to its delayed release, a single-dose cross-over comparative bioavailability study in healthy volunteers was conducted.

The study was a single center, randomized, single dose, laboratory-blinded, 3-period, 3-sequence, crossover design in 18 healthy male and female subjects. Subjects received either the combination of the regular cholestyramine powder with HCTZ 25 mg, or the combination of the new ECC capsules with HCTZ 25 mg, or HCTZ 25 mg alone, as the victim drug. The test treatment consisted of ten (10) capsules of ECC per subject (total 4.25 g cholestyramine), administered as a single dose concomitantly with 25 mg HCTZ (immediate release tablet). The following 3 treatments were administered in each study period according to the randomization scheme
Treatment-A: A single 4.25 g dose (10×425 mg Enteric-coated (EC) capsule) administered 30 mins prior to a single 25 mg dose (1×25 mg tablet) of pms-HCTZ
Treatment-B: Reference 1—Olestyr® (cholestyramine resin) for oral suspension USP (Orange flavor-Regular)—powder administered as a single 4 g dose (1×9 g sachet) administered 30 mins prior to a single 25 mg dose (1×25 mg tablet) of pms-HCTZ
Treatment-C: Reference 2—A single 25 mg dose (1×25 mg tablet) of pms-HCTZ taken alone, 30 mins after administration of water
   The treatments were administered according to the following table.

TABLE 59
Study Sequences

| | Period 1 | Period 2 | Period 3 |
|---|---|---|---|
| Sequence ABC (n = 6) | Treatment-A | Treatment-B | Treatment-C |
| Sequence CAB (n = 6) | Treatment-C | Treatment-A | Treatment-B |
| Sequence BCA (n = 6) | Treatment-B | Treatment-C | Treatment-A |

Treatment-A:
A single 4.25 g dose (10 capsules of 425 mg) of the Test was first administered with approximately 180 mL of water at ambient temperature. Subjects were instructed to swallow the capsules whole, without chewing or breaking the capsules.

Treatment-B:
A single 4 g dose of the Reference-1 was first administered as follows: the content of one pouch was placed in 120 mL of water at ambient temperature and was mixed thoroughly and the subjects were asked to drink the prepared mixture. Thereafter, the glass was rinsed with approximately 60 mL of water at ambient temperature, for a total of approximately 180 mL of water administered. Thirty (30) mins later, a single 25 mg dose of the Reference-2 was administered with approximately 240 mL of water at ambient temperature.

Treatment-C:
Thirty (30) mins prior to the Reference-2 administration, approximately 180 mL of water at ambient temperature was consumed. Thirty (30) mins later, a single dose of 25 mg of the Reference-2 was administered with approximately 240 mL of water at ambient temperature.

A total of 18 subjects were included in this study and, after randomization, 17 subjects (94%) received Treatment-A, all 18 subjects received Treatment-B and all 18 subjects Treatment-C. One subject (6%) withdrew consent from the study; 17 subjects (94%) completed the study.

The primary objective of this study was to show the absence of drug interaction between HCTZ and the new cholestyramine EC capsule compared to cholestyramine regular powder following a single oral dose administration under fasting conditions.

Treatment-A (HCTZ Together with EC Cholestyramine Capsules) Versus Treatment-C(HCTZ Alone)

Mean plasma concentration time courses of hydrochlorothiazide slightly differed after treatment of HCTZ together with EC cholestyramine capsules (Treatment-A) and HCTZ alone (Treatment-C) with peak concentrations of HCTZ reaching slightly earlier after Treatment-A when compared to Treatment-C.

As per Health Canada requirements, the Treatment-A to Treatment-C ratio of geometric LSmeans and corresponding 90% CIs for Cmax and AUC0-T were 90.9% (CI: 79.7-103.6%) and 75.9% (CI: 67.0-86.0%), respectively.

As per FDA requirements, the Treatment-A to Treatment-C ratio of geometric LSmeans and corresponding 90% CIs for Cmax, AUC0-T and AUC0-∞ were 90.90% (CI: 79.75-103.62%), 75.89% (CI: 66.95-86.02%) and 75.72% (CI: 67.19-85.34%), respectively.

The statistical results for Treatment-A versus C, indicate that the ratio of geometric LS means with corresponding lower limit of 90% confidence interval are below the predefined acceptance range for AUC0-T and AUC0-∞ (for FDA), whereas for Cmax the geometric LS means ratio falls within the acceptance range, however the lower limit of 90% confidence interval does not meet the acceptance criteria.

Treatment-B (HCTZ Together with Cholestyramine Powder) Versus Treatment-C(HCTZ Alone)

Mean plasma concentration time courses of HCTZ differed after treatment of HCTZ together with cholestyramine powder (Treatment-B) and HCTZ alone (Treatment-C) with slight delay in reaching peak concentrations of HCTZ after Treatment-B when compared to Treatment-C.

As per Health Canada requirements, the Treatment-B to Treatment-C ratio of geometric LSmeans and corresponding 90% CIs for Cmax and AUC0-T were 38.0% (CI: 33.4-43.2%) and 37.0% (CI: 32.7-41.8%), respectively.

As per US FDA requirements, the Treatment-B to Treatment-C ratio of geometric LSmeans and corresponding 90% CIs for Cmax, AUC0-T and AUC0-∞ were 38.00% (CI: 33.43-43.20%), 37.00% (CI: 32.73-41.83%) and 39.49% (CI: 35.13-44.40%), respectively.

For this comparison of interest, the ratios of geometric LS means and corresponding 90% confidence interval were below the predefined acceptance range for Cmax AUC0-T and AUC0-∞.

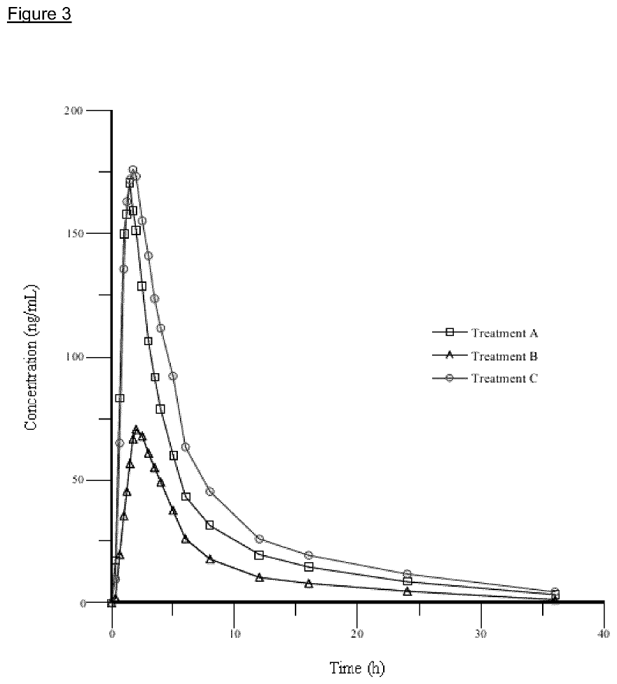

The Linear Profile of Mean Plasma Concentrations versus Time for Hydrochlorothiazide (n=17 for Treatment-A and n=18 for Treatment-B and -C)−(TPD and FDA) is shown in FIG. 3.

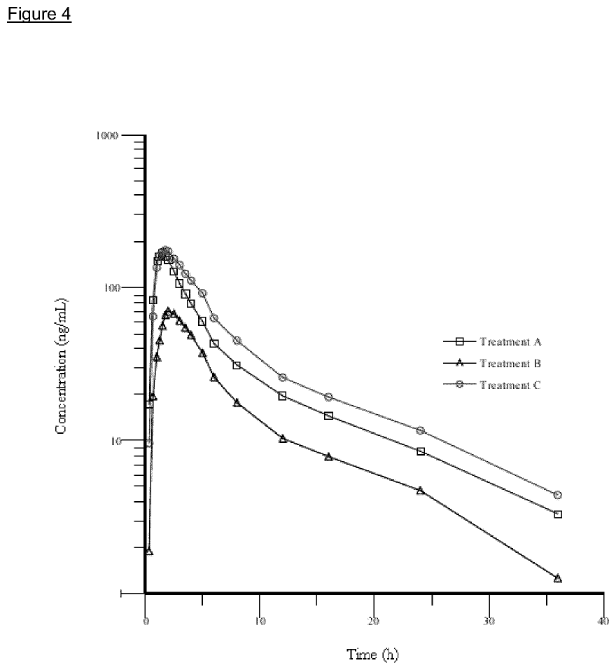

The Logarithmic Profile of the Mean plasma Concentration versus Time for Hydrochlorothiazide (n=17 for Treatment-A and n=18 for Treatment-B and -C)−(TPD and FDA) is shown in FIG. 4.

In conclusion, according to the pre-defined no effect criteria, the results from both comparisons are indicative of an effect of cholestyramine formulations on the pharmacokinetics of HCTZ under fasting conditions. That said, it is important to note that the extent of interaction of both formulations with HCTZ was considerably different. Co-administration of EC cholestyramine capsule lead to a 9.1%, 24.1% and 24.3% reduction in HCTZ Cmax, AUC0-T and AUC0-∞ (for FDA), respectively, whereas co-administration of cholestyramine powder lead to a 62.0%, 63.0% and 60.5% reduction in HCTZ Cmax, AUC0-T and AUC0-∞ (for FDA), respectively.

Furthermore, on comparing the geometric mean ratios between the two different comparisons, it was observed that there is an approximate 2.4-fold, 2.1-fold and 1.9 fold difference in Cmax, AUC0-T and AUC0-∞ (for FDA) ratios, respectively indicating a considerable difference between the interaction potential of the cholestyramine powder and EC capsule of cholestyramine when administered 30 mins prior to HCTZ.

Based on these observations, it can be concluded that although both formulations of cholestyramine (EC capsule and powder) exhibit an effect on the fasting pharmacokinetics of HCTZ, the impact of the EC formulation of cholestyramine is notably lower than the impact of the cholestyramine powder on HCTZ and therefore is expected to be of minor clinical significance compared to that of the non-enteric-coated powder formulation.

The results suggest that the delivery of cholestyramine in a more distal intestinal segment via an enteric-coated capsule, successfully reduces the magnitude of DDIs.

### Example 8—A Phase IIa, Proof of Concept, Randomized, Double-Blind, Dose-Finding, Cross-Over Study of the Efficacy, Safety and Tolerability of a New Enteric-Coated Cholestyramine Capsule in Adult Short Bowel Syndrome Patients

The clinical safety and efficacy of this new formulation of cholestyramine is investigated in a clinical development program involving adult patients with SBS in a Phase IIa proof of concept, randomized, double-blind, dose finding, cross-over study of the efficacy, safety and tolerability of ECC capsules in adult SBS patients. The primary objective of this multicentre study is to investigate the clinical efficacy of new ECC capsules and select the most effective dose in adult patients with SBS. The secondary objectives of this study are to evaluate the safety and tolerability of new ECC capsules in adult patients with SBS, and to evaluate the patients' experience of related symptoms using a Visual Analog Scale. The primary endpoint is the change in the weekly frequency of bowel movements, measured between baseline and the second week of treatment (ie. Days 8 to 14 and Days 36 to 42). Efficacy is assessed as the overall difference vs baseline as well as the difference between the two treatment doses.

This is achieved with a double dummy, 2-period, 2-sequence crossover design where 18 patients are randomized to either “low” dose ECC (1.7 g+matching placebo), or “high” dose ECC (4.25 g) to be taken BID for 14 days per period. A 14-day washout separate the 2 periods. Efficacy is assessed as the overall difference vs baseline as well as the difference between the two treatments in terms of the change in the weekly frequency of bowel movements measured between baseline and the second week of treatment. Changes in the daily stool form score according to the Bristol Stool Form Scale are evaluated.

The following 2 treatments are administered in each study period according to the randomization scheme:
Treatment A: ECC at the 1.7 g daily dose, administered BID as 2 capsules of ECC, plus 3 capsules of placebo, at least 30 mins before breakfast and 2 capsules of ECC, plus 3 capsules of placebo at least 30 mins before evening meal.
Treatment B: ECC at the 4.25 g daily dose, administered BID as 5 capsules of ECC at least 30 mins before breakfast and 5 capsules of ECC at least 30 mins before evening meal.
All 18 patients receive both ECC doses by the end of the study.

TABLE 60
Randomization

| | Period 1 | Period 2 |
|---|---|---|
| Sequence AB (n = 9) | Treatment A | Treatment B |
| Sequence BA (n = 9) | Treatment B | Treatment A |

While the aspects described herein have been described in conjunction with the example aspects outlined above, various alternatives, modifications, variations, improvements, and/or substantial equivalents, whether known or that are or may be presently unforeseen, may become apparent to those having at least ordinary skill in the art. Accordingly, the example aspects, as set forth above, are intended to be illustrative, not limiting. Various changes may be made without departing from the spirit and scope of the disclosure. Therefore, the disclosure is intended to embrace all known or later-developed alternatives, modifications, variations, improvements, and/or substantial equivalents.

Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”

1-89. (canceled)

90. A unit dosage form comprising a capsule having a coating comprising one or more enteric polymers and a capsule fill comprising cholestyramine,
wherein the capsule has the following in vitro dissolution profile using a USP Type 3 apparatus: (a) pH 1.2, no disintegration of capsule for 1 hour; and (b) pH 6 to 7, preferably 6.2 to 7, more preferably 6.5 to 6.8, or 6.5, 6.6, 6.7 or 6.8 bursting of capsule and dispersion of the capsule contents within 30 minutes, wherein (a) is performed prior to (b), or
wherein the capsule has the following in vitro dissolution profile using a USP Type 3 apparatus: (a) pH 1.2: no disintegration of capsule for 1 hour; (b) pH 6.2-7, preferably 6.5-6.8, preferably 6.5 or 6.8: bursting of capsule and dispersion of the capsule content within 30 mins, and (c) pH 6.0, bursting of capsule and dispersion of capsule content after at least 45 mins, wherein (a) is performed prior to (b) or (c), or having the following in vitro dissolution profile using a USP Type 3 apparatus: (a) pH 1.2: no disintegration of capsule for 1 hour; (b) pH 6.8: bursting of capsule and dispersion of the capsule content within 20 mins, preferably within 15 mins; and (c) pH 6.0, nor bursting or bursting of capsule and dispersion of capsule content after at least 45 mins, wherein (a) is performed prior to (b) or (c).

91. The unit dosage form of claim 90,
wherein the unit dosage form starts releasing the capsule fill at pH>6, preferably at pH>6.2, or
wherein in vivo the unit dosage form starts releasing the capsule fill after the proximal part of the small intestine, or
wherein in vivo the unit dosage form releases the capsule fill in the distal part of the small intestine, preferably in the jejunum, more preferably in the ileum.

92. The unit dosage form of claim 90,
wherein the capsule fill comprises at least 70% w/w cholestyramine, more preferably at least 75% w/w cholestyramine, more preferably at least 80% w/w cholestyramine, even more preferably at least 83% w/w cholestyramine, and most preferably at least 85% w/w cholestyramine, or
wherein the amount of cholestyramine in the unit dosage form ranges from 350 to 500 mg, more preferably 400 to 450 mg, most preferably 425 mg.

93. The unit dosage form of claim 90, wherein the capsule comprises gelatin, agar, xanthan gum, karaya gum, locust bean gum, gum arabic, pullulan, methyl cellulose, hydroxyethyl cellulose, hydroxypropyl methylcellulose, sodium alginate, or combinations thereof, preferably HPMC.

94. The unit dosage form of claim 90, wherein the capsule is a size 0, 00 or 000 capsule, more preferably size 00.

95. The unit dosage form of claim 90, wherein the capsule fill comprises, consists essentially of, or consists of from 80 to 90% w/w, preferably 83 to 85% w/w cholestyramine, from 10 to 20% w/w of a bulking agent, from 0.25 to 2% w/w of a glidant and from 0.25 to 2% w/w of a lubricant, the total of the fill being 100% w/w.

96. The unit dosage form of claim 90, wherein the enteric polymer coating level comprises between 5% and 15%, between 6% and 15% w/w of the UDF, preferably between 5.5% and 10%, most preferably between 5.8% and 6.8%, and even more preferably between 6.0 and 6.5%, or
wherein the enteric polymer coating is an acrylate or acrylic acid polymer or co-polymer, optionally comprising one or more ammonio methacrylate copolymers, or
wherein the enteric polymer coating is in the form of an acrylic resin lacquer in the form of an aqueous dispersion, preferably copolymers of acrylic and methacrylic esters with a low content of quaternary ammonium groups, or
wherein the enteric polymer coating is selected from the group consisting of co-polymers based on polymethacrylic acid and methacrylates, ethyl acrylate and methyl acrylate, co-polymers of acrylic and methacrylic acid esters, hydroxypropyl methylcellulose phthlate, hydroxypropyl methylcellulose acetate succinate, cellulose acetate phthalate, polyvinyl acetate phthalate or mixtures thereof, or
wherein the enteric polymer coating comprises or consists of a methacrylic acid copolymer, preferably Poly(methacrylic acid-co-ethyl acrylate) 1:1.

97. The unit dosage form of claim 90, wherein the enteric polymer coating comprises a film former, a plasticizer and/or an anti-adherent, preferably has the following formulation:

| Components | Function |
|---|---|
| Blend: | |
| Cholestyramine Resin, | Active ingredient |
| Lactose Monohydrate | Bulking agent/filler |
| Colloidal Silicon Dioxide | Glidant |
| Magnesium Stearate | Lubricant |
| Capsule Shell (Cap and Body) Size 00: | |
| Hypromellose | Capsule structure |
| Titanium dioxide | Opacifier |
| Enteric Coat: | |
| Methacrylic Acid Copolymer Dispersion | Control release polymer |
| Plasacryl ™ T20, | Plasticizer |
| Talc | Anti-adherent |

98. The unit dosage form of claim 90,
further comprising a barrier coating between the capsule shell and the enteric polymer coating, preferably a seal coating, or
wherein the barrier coating level preferably comprises 6% to 15% w/w of the unit dosage form, preferably 7% to 12%, and most preferably 9% to 11%, or
wherein the barrier coating level preferably comprises a control release polymer, a plasticizer and/or an anti-adherent, or
wherein the capsule fill, the capsule shell, the enteric coating and/or the barrier coating further comprise excipients such as bulking agents or diluents, glidants, lubricants, and/or other common excipients, or
wherein the bulking agents or diluents further include, for example, dextrose, lactose, glucose, glycine, inositol, mannitol, sorbitol, sucrose, a polyethyleneglycol (PEG), or a polyvinylpyrrolidine (PVP), or a combination thereof, preferably lactose monohydrate, or
wherein the glidants further include, for example, calcium phosphate, calflo E, cellulose (powder), colloidal silicon dioxide, magnesium silicate, magnesium trisilicate, silicon dioxide, starch, talcum powder, or a combination thereof, preferably colloidal silicon dioxide, or
wherein the lubricants further include, for example, magnesium stearate, sodium stearyl fumarate, hydrogenated castor oil, hydrogenated soybean oil, polyethylene glycol, or a combination thereof, preferably magnesium stearate, or
wherein the plasticizer is preferably Triethyl citrate, or
wherein the antiadherent is preferably talc, or
wherein the other common excipients comprises glyceryl monostearate, and/or polysorbate 80, or
wherein (i) the capsule fill further comprises in addition to cholestyramine or consists essentially of a filler, one or more glidant and one or more lubricant; (ii) the barrier coating or seal coat comprises or consists essentially of HPMC, one or more plasticizer and one or more anti-adherent; and/or (iii) the enteric coating comprises or consists essentially of a Methacrylic acid copolymer, one or more plasticizer, and one or more an-anti-adherent, or
wherein the enteric polymer coating and/or the sealing coating are applied onto the capsule by a fluidized bed method, a rotating fluidized bed method, a side-vented pan or coating pan method, preferably a coating pan.

99. The unit dosage form of claim 90, comprising the following components:

| Components | Function |
|---|---|
| Blend: | |
| Cholestyramine Resin, | Active ingredient |
| Lactose Monohydrate | Bulking agent |
| Colloidal Silicon Dioxide | Glidant |
| Magnesium Stearate | Lubricant |
| Capsule Shell (Cap and Body) Size 00: | |
| Hypromellose | Capsule structure |
| Titanium dioxide | Opacifier |
| Seal Coat: | |
| Hypromellose | Film former |
| Triethyl Citrate | Plasticizer |
| Talc, USP | Anti-adherent |
| Enteric Coat: | |
| Methacrylic Acid Copolymer Dispersion | Control release polymer |
| Plasacryl ™ T20, | Plasticizer |
| Talc | Anti-adherent |

100. The unit dosage form of claim 90, wherein the blend has about the following formulation:
a)

| Ingredient | % w/w |
|---|---|
| Cholestyramine | 85.02 |
| Lactose Flowlac 100 | 14.00 |
| Colloidal silicon dioxide | 0.48 |
| Magnesium stearate | 0.50 |

b)

| Ingredient | % w/w |
|---|---|
| Cholestyramine | 88.24 |
| Lactose monohydrate supertablet 11SD EP | 10.78 |
| Colloidal silicon dioxide | 0.48 |
| Magnesium stearate | 0.50 |
| Total | |

c)

| Ingredient | % w/w |
|---|---|
| Cholestyramine | 83.34 |
| Lactose monohydrate supertablet 11SD EP | 15.69 |
| Colloidal silicon dioxide | 0.48 |
| Magnesium stearate | 0.50 |
| Total | 100 |

101. The unit dosage form of claim 90, wherein the seal coating has about the following formulation:
a)

| Ingredients for seal coat | % w/w |
|---|---|
| HPMC E5 LV | 83.33 |
| TEC | 8.33 |
| Talc | 8.33 |
| Total | 100 |

b)

| Ingredients for seal coat (COEP-049) | % w/w |
|---|---|
| Opadry 03K19229 | 80.00 |
| Talc | 20.00 |
| Total | 100 |

c)

| Ingredients for seal coat (COEP-053) | % w/w |
|---|---|
| HPMC E5 LV | 71.43 |
| Triethyl citrate | 7.14 |
| Talc | 21.43 |
| Total | 100 |

or
d)

| Ingredients for seal coat | % w/w |
|---|---|
| HPMC E5 | 90.91% |
| Triethyl citrate | 9.09% |
| Total | 100 |

102. The unit dosage form of claim 90, wherein the enteric coating has about the following formulation:
A)

| Ingredients for enteric coat | % w/w |
|---|---|
| HPMCAS JZ160-122 | 95.0 |
| Triethyl citrate | 5.0 |
| Total | 100 |

B)

| Ingredients for enteric coat | % w/w |
|---|---|
| HPMCAS XCS47163 | 62.11 |
| Triethyl citrate (TEC) | 17.39 |
| Talc | 18.63 |
| Sodium lauryl sulfate (SLS) | 1.86 |
| Total | 100 |

C)

| Ingredients for enteric coat (COEP-051) | % w/w |
|---|---|
| Poly(methyl acrylate-co-methyl methacrylate- | 51.20 |
| co-methacrylic acid) 7:3:1 | |
| Poly(methacrylic acid-co-ethyl acrylate, 1:1 | 12.80 |
| (e.g. Eudragit L30D55) | |
| Triethyl citrate | 4.00 |
| Talc | 32.00 |
| Total | 100 |

D)

| Ingredients for enteric coat (COEP-053) | % w/w |
|---|---|
| Poly(methyl acrylate-co-methyl methacrylate- | 70.16 |
| co-methacrylic acid) 7:3:1 | |
| Poly(methacrylic acid-co-ethyl acrylate, 1:1 | 28.06 |
| (e.g. Eudragit L30D55) | |
| Plasacryl T20 | 1.77 |
| Total | 100 |

E)

| Ingredients for enteric coat (COEP-054) | % w/w |
|---|---|
| Poly(methyl acrylate-co-methyl methacrylate- | 57.9 |
| co-methacrylic acid) 7:3:1 | |
| Poly(methacrylic acid-co-ethyl acrylate, 1:1 | 23.1 |
| (e.g. Eudragit L30D55) | |
| Plasacryl T20 | 6.7 |
| Talc | 12.2 |
| Total | 100 |

F)

| Ingredients for enteric coat (COEP-055) | % w/w |
|---|---|
| Poly(methyl acrylate-co-methyl methacrylate- | 64.91 |
| co-methacrylic acid) 7:3:1 | |
| Poly(methacrylic acid-co-ethyl acrylate, 1:1 | 16.23 |
| (e.g. Eudragit L30D55) | |
| Plasacryl T20 | 6.69 |
| Talc | 12.17 |
| Total | 100 |

G)

| Ingredients for enteric coat (COEP-056) | % w/w |
|---|---|
| Poly(methyl acrylate-co-methyl methacrylate- | 55.96 |
| co-methacrylic acid) 7:3:1 | |
| Poly(methacrylic acid-co-ethyl acrylate, 1:1 | 25.18 |
| (e.g. Eudragit L30D55) | |
| Plasacryl T20 | 6.69 |
| Talc | 12.17 |

H)

| Ingredients for enteric coat (COEP-058) | % w/w |
|---|---|
| Poly(methacrylic acid-co-ethyl acrylate, 1:1 | 80.00 |
| (e.g. Eudragit L30D55) | |
| Plasacryl T20 | 12.00 |
| Talc | 8.00 |
| Total | 100 |
| | COEP-059 | COEP-060 | COEP-061 |
|---|---|---|---|
| Ingredients for enteric coat | % w/w | % w/w | % w/w |
| Poly(methacrylic acid-co-ethyl acrylate, 1:1 | 74.08 | 74.08 | 74.08 |
| (e.g. Eudragit L30D55) | | | |
| Plasacryl T20 | 18.52 | 18.52 | 18.52 |
| Talc | 7.41 | 7.41 | 7.41 |
| Total | 100 | 100 | 100 |

Or
J)

| Ingredients for enteric coat (COEP 071 | |
|---|---|
| (F1 and F2), COEP-072) | % w/w |
| Poly(methacrylic acid-co-ethyl acrylate, 1:1 | 74.2 |
| (e.g., Eudragit L30D55) | |
| Plasacryl T20 | 19.7 |
| Talc | 7.39 |
| Total | 100 |

103. The unit dosage form of claim 90, wherein the formulation is defined as below:

| | | |
|---|---|---|
| | | Phase II trial (Scale |
| Scintigraphy | DDI | up) |

Capsule Core

| | | | | | | |
|---|---|---|---|---|---|---|
| Cholestyramine Resin | 85.00% | (425 mg) | 85.00% | (425 mg) | 85.00% | (425 mg) |
| (active ingredient) | | | | | | |
| Lactose monohydrate | 13.42% | (67.1 mg) | 14.02% | (70.1 mg) | 14.02% | (70.1 mg) |
| (diluent) | | | | | | |
| Colloidal silicon dioxide | 0.48% | (2.4 mg) | 0.48% | (2.4 mg) | 0.48% | (2.4 mg) |
| (glidant) | | | | | | |
| Magnesium stearate | 0.5% | (2.5 mg) | 0.5% | (2.5 mg) | 0.5% | (2.5 mg) |
| (lubricant) | | | | | | |
| | | | |
|---|---|---|---|
| Samarium oxide | 0.6% | — | — |

Sealing coat

| | | | | | | |
|---|---|---|---|---|---|---|
| HPMC (Hypromellose) | 71.43% | (44.5 mg) | 71.43% | (44.5 mg) | 71.43% | (44.5 mg) |
| (film former) | | | | | | |
| Triethyl citrate (TEC) | 7.14% | (4.4 mg) | 7.14% | (4.4 mg) | 7.14% | (4.4 mg) |
| (plasticizer) | | | | | | |
| Talc (Lo-micron) | 21.43% | (13.3 mg) | 21.43% | (13.3 mg) | 21.43% | (13.3 mg) |
| (anti-adherent, filler of | | | | | | |
| gaps) | | | | | | |
| | | | |
|---|---|---|---|
| Seal coating weight gain | 10% | 10% | 10% |

Enteric coating

| | | | | | |
|---|---|---|---|---|---|
| Methacrylic acid | 74.08 | (30.5 mg) | 74.08 | (30.5 mg) | 74.16% |
| copolymer (Eudragit ™ | | | | | |
| L30D-55, 30% solid | | | | | |
| dispersion) | | | | | |
| Plasacryl ™ T20 (20% | 18.52% | (7.6 mg) | 18.52% | (7.6 mg) | 18.42% |
| solid dispersion) | | | | | |
| Talc (Lo-micron) | 7.41% | (3.0 mg) | 7.41% | (3.0 mg) | 7.42% |
| (anti-adherent) | | | | | |
| | | | |
|---|---|---|---|
| Enteric coating weight | 6.5% | 6.0% | |
| gain (%) | | | |
| Enteric coating weight | 6.5% | 6.0% | 6.5% |
| gain | | | |

104. A method of treating diarrhea, comprising administering to a patient the unit dosage form as defined in claim 90,
wherein the diarrhea is preferably bile acid diarrhea (BAD) or chronic diarrhea, or
wherein the diarrhea is preferably associated with Bile acid malabsorption (BAM), Short bowel syndrome (SBS), ileal resection, steatorrhea (fat stool), Crohn's disease, vagotomy, diabetic vagal neuropathy, radiation and/or pruritus in patients with cholestasis, more preferably with Short bowel syndrome (SBS), or
wherein the diarrhea is preferably associated with Type I Secondary BAM (such as ileal resection, ileal dysfunction, impaired reabsorption, e.g. Crohn's disease or necrotizing enterocolitis), Type II Primary BAM (such as IBS-D), or Type III miscellaneous associated BAM disorders (such as post-cholecystectomy, gastric surgery, chronic pancreatitis, celiac disease, SIBO, radiation, microscopic colitis), or
a method of treating hypercholesterolemia, type II hyperlipoproteinemia and/or type 2 diabetes mellitus, comprising administering to a patient the unit dosage form as defined in claim 1, or
a method of treating pruritus in a patient with cholestasis, comprising administering to said patient the unit dosage form as defined in claim 1, or a method for reducing or eliminating drug interactions with cholestyramine, such as a blood thinner like warfarin (Coumadin, Jantoven); digoxin (digitalis, Lanoxin); propranolol (Inderal); a diuretic such as hydrochlorothiazide (HCTZ); thyroid hormones such as levothyroxine (Synthroid, Levoxyl, Levothroid); birth control pills or hormone replacement; seizure medicines such as phenytoin (Dilantin) and phenobarbital (Luminal, Solfoton); an antibiotic such as amoxicillin (Amoxil, Trimox, others), doxycycline (Adoxa, Doryx, Oracea, Vibramycin), minocycline (Dynacin, Minocin, Solodyn, Vectrin), penicillin (BeePen-VK, Pen-Vee K, Veetids, others), tetracycline (Brodspec, Panmycin, Sumycin, Tetracap); aspirin; duloxetine (Cymbalta); fish oil (omega-3 PUFAs); pregabalin (Lyrica); alprazolam (Xanax); acetaminophen; and/or vitamins such as Vitamin B12 (cyanocobalamin); Vitamin C; Vitamin D3; Vitamin A, Vitamin E; Vitamin K, comprising administering to a patient the unit dosage form as defined in claim 1, or
a method for reducing or eliminating drug interactions with cholestyramine, such as estrogens, thiazide diuretics, digoxin and related alkaloids, loperamide, phenylbutazone, barbiturates, thyroid hormones, warfarin and/or some antibiotics comprising administering to a patient who is receiving concomitant administration of a medication which is known to interact with conventional orally-administered cholestyramine, the unit dosage form as defined in claim 1, or
a method for preventing macronutrients and/or micronutrients absorption deficiency in the gastrointestinal system of a patient, comprising administering to a patient the unit dosage form as defined in claim 1, wherein the macronutrients and the micronutrients preferably include fat and fat-soluble vitamins.

105. The method of claim 104, wherein the daily dose of cholestyramine is 425 mg to 30 g, preferably 850 mg to 12 g, most preferably 1700 mg to 8.5 g or
wherein the daily dose of cholestyramine is 425 mg to 4250 mg/day, preferably 425 mg to 3400 mg/day, more preferably 425 mg/day, 850 mg/day, 1275 mg/day, 1700 mg/day, 2125 mg/day, 2550 mg/day, 2975 mg/day, 3400 mg/day, 3825 mg/day or 4250 mg/day, or ranges between any one of these amounts.

106. A process for making the unit dosage form as defined in claim 90, comprising
1) blending of cholestyramine
2) encapsulating and
3) enteric coating.

107. The process of claim 106, preferably further comprising a step of sealing the capsule before the step of enteric coating for making the unit dosage form as defined in claim 1, or
wherein the process is for large scale manufacture, or
wherein the steps of sealing the capsule and of enteric coating comprise pan coating.
